(19) United States
(12) Patent Application Publication
(10) Pub. No.: US 2014/0057842 A1
(43) Pub. Date: Feb. 27, 2014

(54) COMPOSITIONS AND METHODS FOR CELL HOMING AND ADIPOGENESIS

(71) Applicants: Jeremy J. Mao, Closter, NJ (US); Bhranti Shah, New York, NY (US)

(72) Inventors: Jeremy J. Mao, Closter, NJ (US); Bhranti Shah, New York, NY (US)

(73) Assignee: The Trustees of Columbia University in the City of New York, New York, NY (US)

(21) Appl. No.: 13/877,279

(22) PCT Filed: Oct. 3, 2011
(86) PCT No.: PCT/US11/54658

§ 371 Date: Nov. 11, 2013

Related U.S. Application Data
(60) Provisional application No. 61/388,922, filed on Oct. 1, 2010.

(51) Int. Cl. A61K 38/30; A61K 31/405; A61K 31/506; A61K 31/522; A61K 31/573; A61K 38/18; A61K 38/28

(52) U.S. Cl. CPC A61K 38/30; A61K 38/1825; A61K 31/405; A61K 38/28; A61K 31/522; A61K 31/573; A61K 31/506
USPC 514/6.5; 514/8.6; 514/9.1

(57) ABSTRACT

Provided is a method of causing a cell to migrate to a scaffold and there differentiate to form adipose or adipose-like cells or tissue. Also provided is a method of treating a mammal that has a tissue defect. Further provided is a tissue scaffold comprising a cell homing composition and an adipogenic composition. Additionally, a method of making a tissue scaffold capable of recruiting a cell and differentiating the recruited cell to form adipose or adipose-like cells or tissue is provided.

### CROSS-REFERENCE TO RELATED APPLICATIONS

The present application claims the benefit of U.S. Provisional Application Ser. No. 61/388,922 filed Oct. 1, 2010, which is incorporated herein by reference in its entirety.

### STATEMENT REGARDING FEDERALLY SPONSORED RESEARCH OR DEVELOPMENT

This invention was made with government support under R01EB06362 and RC2DE020767 awarded by the National Institutes of Health. The government has certain rights in the invention.

### MATERIAL INCORPORATED-BY-REFERENCE

Not Applicable.

### FIELD OF THE INVENTION

The present invention generally relates to generation and regeneration of adipose tissue.

### BACKGROUND OF THE INVENTION

Adipose tissue is in critical demand for reconstruction of soft tissue wounds, breast cancer defects, facial defects, lipoatrophy and for soft tissue augmentation. Clinically, autologous fat transfer is in common practice. Autologous tissue grafts are harvested from one part of the patient's body for the reconstruction of another part. Key drawbacks to this technique include donor site morbidity and volume loss over time. Volume reduction after autologous fat transfer can be as high as 70%. Experimentally, adipose and bone-marrow stem cells or preadipocytes have been grafted in natural or synthetic materials for adipose tissue regeneration (see e.g., Gomillion and Burg 2006 Biomaterials 6052-6063). Large quantities of stem/progenitor cells are, however, typically required but can be scarce in patients.

### SUMMARY OF THE INVENTION

Teachings of the present disclosure include a method of forming adipose tissue.

One aspect provides a method of forming adipose tissue. In some embodiments, the method includes providing a scaffold. In some embodiments, the scaffold is placed in fluid communication with a progenitor cell. In some embodiments, the progenitor cell is induced to migrate into or onto the scaffold. In some embodiments, the progenitor cell is induced to form an adipose cell or adipose-like cell while in or on the scaffold.

In some embodiments, the scaffold includes an effective amount of a cell homing composition. In some embodiments the scaffold includes an adipogenic composition. In some embodiments, the scaffold does not comprise a transplanted cell.

Another aspect provides a method of treating a subject having a soft tissue defect. In some embodiments, the method of treating a subject having a soft tissue defect includes implanting a scaffold into a subject in need thereof. In some embodiments, the scaffold includes an effective amount of a cell homing composition. In some embodiments, the effective amount of the cell homing composition is an amount which induces migration of a progenitor cell into or onto the scaffold. In some embodiments the scaffold includes an adipogenic composition. In some embodiments, the adipogenic composition induces formation of an adipose cell or an adipose-like cell from a progenitor cell. In some embodiments, the scaffold does not include a transplanted cell prior to implantation in the subject.

Another aspect provides a soft tissue construct. In some embodiments, the construct includes a scaffold having an effective amount of a cell homing composition. In some embodiments, the effective amount of cell homing composition is an amount which induces migration of a progenitor cell into or onto the scaffold. In some embodiments, the construct includes a scaffold having an effective amount of an adipogenic composition. In some embodiments, the effective amount of the adipogenic composition induces formation of an adipose cell or an adipose-like cell from a progenitor cell. In some embodiments, the scaffold does not include a transplanted cell prior to implantation in the subject. In some embodiments, the scaffold is in fluid communication with a progenitor cell. In some configurations, where the scaffold is in fluid communication with the progenitor cell, the effective amount of the cell homing composition can induce migration of a progenitor cell into or onto the scaffold.

In some embodiments, the cell homing composition can include insulin-like growth factor 1 (IGF1). In some configurations, the IGF1 can be at a ratio of about 0.1/250 to about 250/250 (μg IGF1 per mg scaffold). In some embodiments, the cell homing composition can include basic fibroblast growth factor (bFGF). In some configurations, the bFGF can be at a ratio of about 0.1/250 to about 250/250 (μg bFGF per mg scaffold). In some embodiments, the cell homing composition can include both IGF1 and bFGF. In some configurations, the IGF1 can be at a ratio of about 0.1/250 to about 250/250 (μg IGF1 per mg scaffold) and the bFGF can be at a ratio of about 0.1/250 to about 250/250 (μg bFGF per mg scaffold).

In some embodiments, a secretase γ inhibitor is included. In some embodiments, the scaffold includes a secretase γ inhibitor. In some embodiments, the cell homing composition includes a secretase γ inhibitor. In some embodiments, the adipogenic composition includes a secretase γ inhibitor. In some configurations, the secretase γ inhibitor can be provided in an amount effect to reduce, substantially reduce, or eliminate inhibition of adipogenesis by an EGF receptor comprised by the progenitor cell. In some configurations, the secretase γ inhibitor can have a concentration of about 1.0 μM to about 100 μM. In some configurations, the secretase γ inhibitor can have a ratio of about 0.1/250 to about 250/250 (μg secretase γ inhibitor per mg scaffold).

In some embodiments, a Notch gamma secretase inhibitor is included. In some embodiments, the scaffold includes a Notch gamma secretase inhibitor. In some embodiments, the cell homing composition includes a Notch gamma secretase inhibitor. In some embodiments, the adipogenic composition includes a Notch gamma secretase inhibitor. In some configurations, the Notch gamma secretase inhibitor can be provided in an amount effect to reduce, substantially reduce, or eliminate inhibition of adipogenesis by an EGF receptor comprised by the progenitor cell. In some configurations, the Notch gamma secretase inhibitor can have a concentration of about 1.0 μM to about 100 μM. In some configurations, the Notch gamma secretase inhibitor can have a ratio of about 0.1/250 to about 250/250 (μg inhibitor per mg scaffold).

In some embodiments, a MAPk inhibitor is included. In some embodiments, the scaffold includes a MAPk inhibitor. In some embodiments, the cell homing composition includes a MAPk inhibitor. In some embodiments, the adipogenic composition includes a MAPk inhibitor. In some configurations, the MAPk inhibitor can be provided in an amount effect to reduce, substantially reduce, or eliminate inhibition of adipogenesis by an EGF receptor comprised by the progenitor cell. In some configurations, the MAPk inhibitor can have a concentration of about 1.0 μM to about 100 μM. In some configurations, the MAPk inhibitor can have a ratio of about 0.1/250 to about 250/250 (μg inhibitor per mg scaffold).

In some embodiments, the adipogenic composition can include one or more of indomethacin, insulin, isobutyl-methylxanthine (IBMX), dexamethasone, or Pyrintegrin. In some configurations, indomethacin is present in a ratio of about 0.1/250 to about 250/250 (mg indomethacin per mg scaffold). In some configurations, insulin is present at a ratio of about 0.1/250 to about 250/250 (mg insulin per mg scaffold). In some configurations, IBMX is present at a ratio of about 0.1/250 to about 250/250 (mg IBMX per mg scaffold). In some configurations, dexamethasone is present at a ratio of about 0.1/250 to about 250/250 (mg dexamethasone per mg scaffold). In some configurations, Pyrintegrin is present at a ratio of about 0.1/250 to about 250/250 (mg Pyrintegrin per mg scaffold).

In some embodiments, the progenitor cell is an adipose tissue derived cell, a pre-adipocyte, a mesenchymal stem cell (MSC), an MSC-derived cell, or an adipocyte. In some embodiments, progenitor cells include one or more of an adipose tissue derived cell, a pre-adipocyte, a mesenchymal stem cell (MSC), an MSC-derived cell, or an adipocyte.

In some embodiments, the scaffold includes a biocompatible matrix material. In some configurations, the scaffold includes poly(lactic-co-glycolic acid) (PLGA). In some configurations, the scaffold includes at least one physical channel.

In some embodiments, progenitor cells are present in the scaffold at a density of about 0.0001 million cells (M) ml⁻¹ to about 1000 M ml⁻¹. In some embodiments, adipose cells or adipose-like cells are present in the scaffold at a density of about 0.0001 million cells (M) ml⁻¹ to about 1000 M ml⁻¹.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a series of images and bar graphs showing adipogenesis in C3H10T1/2 cells cultured for 10 days with a microsphere encapsulated adipogenic cocktail including indomethacin, insulin, 3-isobutyl-1-methylxanthine and dexamethasone. FIG. 1A shows H&E and Oil Red-O staining of negative control (AI), 5 mg (AII), 10 mg (AIII), 15 mg (AIV), 20 mg (AV) microspheres and positive control (AVI). FIG. 1B shows percentage of cells differentiated into adipocytes. FIG. 1C shows lipid accumulation detected using Oil Red-O. *p<0.05**p<0.01***p<0.005. Further details regarding methodology are available in Example 1.

FIG. 2 is a series of images showing tissue sections from scaffolds with different combinations of C3H10T1/2 cells and microspheres placed in the lower abdominal subcutaneous fat pad of obese C57BL/6NHsd mice for two weeks. FIG. 2A shows empty scaffold. FIG. 2B shows scaffold with 500K C3H10T1/2 cells. FIG. 2C shows scaffolds with 5 mg adipogenic microspheres. FIG. 2D shows scaffolds with 5 mg adipogenic microspheres and 500K C3H10T1/2 cells. FIG. 2E shows scaffolds with 2.5 mg IGF1 microspheres. FIG. 2F shows scaffolds with 2.5 mg IGF-1 microspheres and 500K C3H10T1/2 cells. ×20 magnification. Further details regarding methodology are available in Example 1.

FIG. 3 is a series of images showing scaffolds with different combinations of C3H10T1/2 cells and microspheres placed in the lower abdominal subcutaneous fat pad of obese C57BL/6NHsd mice for two weeks. FIG. 3A shows empty scaffold. FIG. 3B shows scaffold with 500K C3H10T1/2 cells. FIG. 3C shows scaffolds with 5 mg adipogenic microspheres. FIG. 3D shows scaffolds with 5 mg adipogenic microspheres and 500K C3H10T1/2 cells. FIG. 3E shows scaffolds with 2.5 mg IGF1 microspheres.

FIG. 3F shows scaffolds with 2.5 mg IGF1 microspheres and 500K C3H10T1/2 cells. ×40 magnification. Further details regarding methodology are available in Example 1.

FIG. 4 is a line and scatter plot showing change over 28 days in PPARγ expression of hADSCs treated with control medium; adipogenic differentiation medium (ADM); ADM plus 10 μM of Notch gamma Secretase Inhibitor (Inh1) (ADM+Inh1); ADM plus 10 μM of MAPK Inhibitor (Inh2) (ADM+Inh2); and ADM plus 10 μM of Inh1 and Inh2 (ADM+Inh1, 2).

FIG. 5 is a line and scatter plot showing change over 28 days in C/EBPα expression of hADSCs treated with control medium; adipogenic differentiation medium (ADM); ADM plus 10 μM of Notch gamma Secretase Inhibitor (Inh1) (ADM+Inh1); ADM plus 10 μM of MAPK Inhibitor (Inh2) (ADM+Inh2); and ADM plus 10 μM of Inh1 and Inh2 (ADM+Inh1, 2).

FIG. 6 is a series of brightfield images produced four weeks post-treatment of hADSCs with: (FIG. 6A) ADM; (FIG. 6B) ADM+Inh1; (FIG. 6C) ADM+Inh2; and (FIG. 6D) ADM+Inh1, 2.

FIG. 7 is a bar graph showing Adiponectin content measured in hADSCs treated with control medium; adipogenic differentiation medium (ADM); ADM plus 10 μM of Notch gamma Secretase Inhibitor (Inh1) (ADM+Inh1); ADM plus 10 μM of MAPK Inhibitor (Inh2) (ADM+Inh2), and ADM plus 10 μM of Inh1 and Inh2 (ADM+Inh1, 2) at two and four weeks post-treatment.

FIG. 8 is a bar graph showing Leptin content measured in hADSCs treated with control medium; adipogenic differentiation medium (ADM); ADM plus 10 μM of Notch gamma Secretase Inhibitor (Inh1) (ADM+Inh1); ADM plus 10 μM of MAPK Inhibitor (Inh2) (ADM+Inh2); and ADM plus 10 μM of Inh1 and Inh2 (ADM+Inh1, 2) at two and four weeks post-treatment.

FIG. 9 is a line and scatter plot of PPARγ expression measured for 28 days in hADSCs treated with control medium; adipogenic differentiation medium (ADM); and ADM plus 2 μM of Pyrintegrin (ADM+Pyrintegrin).

FIG. 10 is a line and scatter plot of C/EBPα expression measured for 28 days in hADSCs treated with control medium; adipogenic differentiation medium (ADM); and ADM plus 2 μM of Pyrintegrin (ADM+Pyrintegrin).

FIG. 11 is a pair of images showing lipid staining performed four weeks post-treatment of hADSCs with (FIG. 11A) ADM; and (FIG. 11B) ADM plus Pyrintegrin (ADM+Pyrintegrin).

FIG. 12 is a bar graph showing Adiponectin content measured in hADSCs treated with control medium; adipogenic differentiation medium (ADM); and ADM plus 2 μM of Pyrintegrin (ADM+Drug) at two and four weeks post-treatment.

FIG. 13 is a bar graph showing Adiponectin content measured in hADSCs treated with: control medium; adipogenic differentiation medium (ADM); and ADM plus 2 μM of Pyrintegrin (ADM+Drug) at two and four weeks post-treatment.

FIG. 14 is a bar graph showing Leptin content measured in hADSCs treated with: control medium; adipogenic differentiation medium (ADM); and ADM plus 2 μM of Pyrintegrin (ADM+Drug) at two and four weeks post-treatment.

FIG. 15 is a bar graph showing Glycerol content measured in hADSCs treated with: control medium; adipogenic differentiation medium (ADM); and ADM plus 2 μM of Pyrintegrin (ADM+Drug) at two and four weeks post-treatment.

FIG. 16 is a series of images showing Western blot analysis performed 1-hour post-treatment in hADSCs treated with control medium (Control); adipogenic differentiation medium (ADM); ADM plus 2 μM of Pyrintegrin (ADM+Drug); and Pyrintegrin alone (Drug).

FIG. 17 is a series of images showing Western blot analysis performed 1-hour post-treatment in hADSCs treated with: control medium (Control); adipogenic differentiation medium (ADM); ADM plus 2 μM of Pyrintegrin (ADM+Drug); and Pyrintegrin alone (Drug).

### DETAILED DESCRIPTION OF THE INVENTION

The present disclosure is based at least in part on the observation that homing of host endogenous cells can act as cell sources for adipose tissue regeneration in vivo. As shown herein, in vitro and in vivo experiments show that PLGA scaffolds imbued with a mixture of various adipogenic factors can promote adipogenesis. Further, scaffolds with growth/homing factors were able to home cells into the scaffold. Thus, a combination of an adipogenic cocktail and various homing or angiogenic factors can provide both adipogenesis and cell homing within the same scaffold. Results described herein support the efficacy of adipose regeneration by cell homing, an approach at least equally effective as, and in many ways more beneficial than, cell transplantation.

Regeneration of adipose tissue by cell homing can act as an alternative or adjunctive approach for soft tissue reconstruction or augmentation. Compared with autologous tissue grafts, one of the key advantages of cell-homing based therapies is to minimize donor site morbidity.

Cell homing offers a number of advantages over cell transplantation for soft tissue reconstruction and/or augmentation (see generally Mao et al. 2010 Tissue Engineering Part B: Reviews 16(2), 257-262).

Induced homing of host endogenous cells can overcome key scientific, technical, commercialization, and regulatory issues associated with cell transplantation, such as potential contamination, excessive cost, immunorejection, pathogen transmission, and a lack of training of current clinicians to handle cells. Bioactive cues for cell homing, such as cytokines or chemokines, can be readily packaged and delivered for use in a single procedure, as opposed to frequent multiple procedures in association with cell transplantation. There exists previous regulatory approval for cytokine and chemokine delivery. A cell homing approach benefits from easier clinical delivery of packaged and stored molecular delivery products. A cell homing approach maximizes the body's own regenerative capacity.

Cell homing involves active recruitment of endogenous cells, including stem/progenitor cells, into an anatomic compartment. Tissue regeneration by cell homing can be performed using a biomaterial scaffold in the shape of the tissue of interest and containing a variety of chemoattractants to recruit specific cells into the biomaterial forming the tissue.

Provided is a method for homing cells into a scaffold in conjunction with promotion of adipogenesis. In various embodiments, a controlled release adipogenic composition and a controlled release cell homing composition are introduced into a scaffold or matrix material. The scaffold can be incubated in vitro, ex vivo, or in vivo. The cell homing composition can increase migration of cells, including progenitor cells, into the scaffold or matrix material. The adipogenic composition can promote differentiation of cells, such as progenitor cells, to adipose or adipose-like cells or tissue.

Cell Homing Composition

Various embodiments described herein employ a cell homing composition for promotion of migration of cells. For example, a controlled release cell homing composition can be included in a scaffold so as to promote migration of cells into or onto the scaffold. As another example, a controlled release cell homing composition can be included in a scaffold so as to promote migration of cells into or onto the scaffold which are then induced by an adipogenic composition to differentiate adipose or adipose-like cells.

The cell homing composition can include one or more of insulin-like growth factor 1 (IGF1) or basic fibroblast growth factor (bFGF). For example, the cell homing composition can include IGF1. As another example, the cell homing composition can include bFGF. As another example, the adipogenic composition can include IGF1 and bFGF.

IGF1 can be included in a cell homing composition. In some embodiments, IGF1 can be encapsulated in a microsphere. For example, IGF1 can be encapsulated in a microsphere at a ratio of about 0.1/250 to about 250/250 (μg IGF1 per mg microsphere material). For example, IGF1 can be encapsulated in a microsphere at a ratio of about 0.1/250; about 0.5/250; about 1/250; about 2/250; about 3/250; about 4/250; about 5/250; about 6/250; about 7/250; about 8/250; about 9/250; about 10/250; about 11/250; about 12/250; about 13/250; about 14/250; about 15/250; about 16/250; about 17/250; about 18/250; about 19/250; about 20/250; about 25/250; about 30/250; about 35/250; about 40/250; about 45/250; about 50/250; about 60/250; about 70/250; about 80/250; about 90/250; about 100/250; about 150/250; about 200/250; or about 250/250 (μg IGF1 per mg microsphere material). As another example, IGF1 can be encapsulated in a microsphere at a ratio of about 10 μg/250 mg of microsphere material (see Example 1).

bFGF can be included in a cell homing composition. In some embodiments, bFGF can be encapsulated in a microsphere. For example, bFGF can be encapsulated in a microsphere at a ratio of about 0.1/250 to about 250/250 (μg bFGF per mg microsphere material). For example, bFGF can be encapsulated in a microsphere at a ratio of about 0.1/250; about 0.5/250; about 1/250; about 2/250; about 3/250; about 4/250; about 5/250; about 6/250; about 7/250; about 8/250; about 9/250; about 10/250; about 11/250; about 12/250; about 13/250; about 14/250; about 15/250; about 16/250; about 17/250; about 18/250; about 19/250; about 20/250; about 25/250; about 30/250; about 35/250; about 40/250; about 45/250; about 50/250; about 60/250; about 70/250; about 80/250; about 90/250; about 100/250; about 150/250; about 200/250; or about 250/250 (μg bFGF per mg microsphere material). As another example, bFGF can be encapsulated in a microsphere at a ratio of about 10 μg/250 mg of microsphere material (see Example 1).

As discussed further below, a secretase γ inhibitor can be included in the cell homing composition.

Progenitor Cells

Various compositions and methods described herein provide for recruitment of a progenitor cell, inducing migration of a progenitor cell, or inducing differentiation of a progenitor cell. Some embodiments promote migration of progenitor cells into a scaffold or matrix material, induce formation of adipose or adipose-like cells from progenitor cells, or both.

A progenitor cell is a cell that is undifferentiated or partially undifferentiated, and can divide and proliferate to produce undifferentiated or partially undifferentiated cells or can differentiate to produce at least one differentiated or specialized cell. A progenitor cell can be a pluripotent cell, which means that the cell is capable of self-renewal and of trans-differentiation into multiple tissue types upon differentiation. Pluripotent progenitor cells include stem cells, such as embryonic stem cells and adult stem cells. A progenitor cell can be a multipotent cell. A progenitor cell can be self-renewing. For example, the progenitor cell can be a stem cell. As another example, the progenitor cell can be an adult stem cell. In some embodiments, a progenitor cell can differentiate into, or otherwise form, adipocyte cells or adipocyte-like cells. In some embodiments, a progenitor cell can differentiate into, or otherwise form, adipose cells or adipose-like cells. For example, the progenitor cell can be an adipose tissue derived cell, a pre-adipocyte, a mesenchymal stem cell (MSC), an MSC-derived cell, or an adipocyte.

Progenitor cells can be isolated, purified, or cultured by a variety of means known to the art Methods for the isolation and culture of progenitor cells are discussed in, for example, Vunjak-Novakovic and Freshney (2006) Culture of Cells for Tissue Engineering, Wiley-Liss, ISBN-10 0471629359. A progenitor cell can be comprised by, or derived from, an animal, including, but not limited to, mammals, reptiles, and avians, more preferably horses, cows, dogs, cats, sheep, pigs, and chickens, and most preferably human.

In some embodiments, progenitor cells can migrate into a scaffold or matrix material at a density of about 0.0001 million cells (M) ml⁻¹ to about 1000 M ml⁻¹. For example, progenitor cells can migrate into a scaffold or matrix material at a density of about 1 M ml⁻¹, 5 M ml⁻¹, 10 M ml⁻¹, 15 M ml⁻¹, 20 M ml⁻¹, 25 M ml⁻¹, 30 M ml⁻¹, 35 M ml⁻¹, 40 M ml⁻¹, 45 M ml⁻¹, 50 M ml⁻¹, 55 M ml⁻¹, 60 M ml⁻¹, 65 M ml⁻¹, 70 M ml⁻¹, 75 M ml⁻¹, 80 M ml⁻¹, 85 M ml⁻¹, 90 M ml⁻¹, 95 M ml⁻¹, or 100 M ml⁻¹.

Adipogenic Composition

Various embodiments described herein employ an adipogenic composition for promotion of adipogenesis. For example, a controlled release adipogenic composition can be included in a scaffold so as to promote differentiation of cells in or on the scaffold to adipose or adipose-like cells. As another example, a controlled release adipogenic composition can be included in a scaffold so as to promote adipogenic differentiation of cells that migrated into or onto the scaffold in response to a cell homing composition also included in the scaffold. A variety of adipogenic compositions are known in the art (see e.g., Gomillion and Burg 2006 Biomaterials 6052-6063; Poulous et al. 2010 Exp Biol Med 235, 1185-1193).

The adipogenic composition can include one or more of indomethacin, insulin, isobutyl-methylxanthine (IBMX), or dexamethasone. For example, the adipogenic composition can include indomethacin. As another example, the adipogenic composition can include insulin. As another example, the adipogenic composition can include IBMX. As another example, the adipogenic composition can include dexamethasone. As another example, the adipogenic composition can include indomethacin, insulin, IBMX, and dexamethasone.

Indomethacin can be included in an adipogenic composition. In some embodiments, indomethacin can be encapsulated in a microsphere. For example, indomethacin can be encapsulated in a microsphere at a ratio of about 0.1/250 to about 250/250 (mg indomethacin per mg microsphere material). For example, indomethacin can be encapsulated in a microsphere at a ratio of about 0.1/250; about 0.5/250; about 1/250; about 2/250; about 3/250; about 4/250; about 5/250; about 6/250; about 7/250; about 8/250; about 9/250; about 10/250; about 11/250; about 12/250; about 13/250; about 14/250; about 15/250; about 16/250; about 17/250; about 18/250; about 19/250; about 20/250; about 25/250; about 30/250; about 35/250; about 40/250; about 45/250; about 50/250; about 60/250; about 70/250; about 80/250; about 90/250; about 100/250; about 150/250; about 200/250; or about 250/250 (mg indomethacin per mg microsphere material). As another example, indomethacin can be encapsulated in a microsphere at a ratio of about 5.15 mg/250 mg of microsphere material (see Example 1).

Insulin can be included in an adipogenic composition. In some embodiments, insulin can be encapsulated in a microsphere. For example, insulin can be encapsulated in a microsphere at a ratio of about 0.1/250 to about 250/250 (mg insulin per mg microsphere material). For example, indomethacin can be encapsulated in a microsphere at a ratio of about 0.1/250; 0.2/250; 0.3/250; 0.4/250; 0.5/250; 0.6/250; 0.7/250; 0.8/250; 0.9/250; about 1/250; about 2/250; about 3/250; about 4/250; about 5/250; about 6/250; about 7/250; about 8/250; about 9/250; about 10/250; about 15/250; about 20/250; about 25/250; about 30/250; about 35/250; about 40/250; about 45/250; about 50/250; about 60/250; about 70/250; about 80/250; about 90/250; about 100/250; about 150/250; about 200/250; or about 250/250 (mg insulin per mg microsphere material). As another example, insulin can be encapsulated in a microsphere at a ratio of about 1 mg/250 mg of microsphere material (see Example 1).

IBMX can be included in an adipogenic composition. In some embodiments, IBMX can be encapsulated in a microsphere. For example, IBMX can be encapsulated in a microsphere at a ratio of about 0.1/250 to about 250/250 (mg IBMX per mg microsphere material). For example, IBMX can be encapsulated in a microsphere at a ratio of about 0.1/250; about 0.5/250; about 1/250; about 2/250; about 3/250; about 4/250; about 5/250; about 6/250; about 7/250; about 8/250; about 9/250; about 10/250; about 11/250; about 12/250; about 13/250; about 14/250; about 15/250; about 16/250; about 17/250; about 18/250; about 19/250; about 20/250; about 25/250; about 30/250; about 35/250; about 40/250; about 45/250; about 50/250; about 60/250; about 70/250; about 80/250; about 90/250; about 100/250; about 150/250; about 200/250; or about 250/250 (mg IBMX per mg microsphere material). As another example, IBMX can be encapsulated in a microsphere at a ratio of about 11.1 mg/250 mg of microsphere material (see Example 1).

Dexamethasone can be included in an adipogenic composition. In some embodiments, dexamethasone can be encapsulated in a microsphere. For example, dexamethasone can be encapsulated in a microsphere at a ratio of about 0.1/250 to about 250/250 (mg dexamethasone per mg microsphere material). For example, dexamethasone can be encapsulated in a microsphere at a ratio of about 0.1/250; about 0.5/250; about 1/250; about 5/250; about 10/250; about 15/250; about 20/250; about 25/250; about 30/250; about 31/250; about 32/250; about 33/250; about 34/250; about 35/250; about 36/250; about 37/250; about 38/250; about 39/; about 40/250; about 41/250; about 42/250; about 43/250; about 44/250; about 45/250; about 46/250; about 47/250; about 48/250; about 49/250; about 50/250; about 60/250; about 70/250; about 80/250; about 90/250; about 100/250; about 150/250; about 200/250; or about 250/250 (mg dexamethasone per mg microsphere material). As another example, dexamethasone can be encapsulated in a microsphere at a ratio of about 39.2 mg/250 mg of microsphere material (see Example 1).

The adipogenic composition can contain an agent that can promote progenitor cell survival. For example, the adipogenic composition can include Pyrintegrin (N-(Cyclopropylmethyl)-4-(4-(6-hydroxy-3,4-dihydroquinolin-1-(2H)-yl)pyrimidin-2-ylamino)benzenesulfonamide). Pyrintegrin can promote stem cell (e.g., hESC) survival through protection of the cell surface protein e-cadherin from damage. In some embodiments, Pyrintegrin can be encapsulated in a microsphere. For example, Pyrintegrin can be encapsulated in a microsphere at a ratio of about 0.1/250 to about 250/250 (mg Pyrintegrin per mg microsphere material). For example, Pyrintegrin can be encapsulated in a microsphere at a ratio of about 0.1/250; about 0.5/250; about 1/250; about 5/250; about 10/250; about 15/250; about 20/250; about 25/250; about 30/250; about 31/250; about 32/250; about 33/250; about 34/250; about 35/250; about 36/250; about 37/250; about 38/250; about 39/250; about 40/250; about 41/250; about 42/250; about 43/250; about 44/250; about 45/250; about 46/250; about 47/250; about 48/250; about 49/250; about 50/250; about 60/250; about 70/250; about 80/250; about 90/250; about 100/250; about 150/250; about 200/250; or about 250/250 (mg Pyrintegrin per mg microsphere material). As shown herein, Pyrintegrin can be effective for inducing PPARγ expression in hADSCs (see e.g., Example 8); inducing C/EBPα expression in hADSCs (see e.g., Example 9); inducing lipid accumulation (see e.g., Example 10); inducing adipogenic differentiation (see e.g., Example 11); enhancing Adiponectin cytokine secretion in hADSCs (see e.g., Example 12); enhancing secretion of Leptin cytokine; (see e.g., Example 13); enhancing secretion of Glycerol (see e.g., Example 14); inhibiting the BMP pathway (see e.g., Example 15); and/or not inhibiting the TGFβ/Activin.

One or more of indomethacin, insulin, isobutyl-methylxanthine (IBMX), dexamethasone, and Pyrintegrin can be combined in an adipogenic composition in various combinations according to, for example, independently selected concentrations listed above.

As discussed further below, a secretase γ inhibitor, a Notch gamma secretase inhibitor, or a MAPk inhibitor can be included in the adipogenic composition.

Adipose Cells

Various embodiments described herein induce formation of adipose or adipose-like cells from progenitor cells. Adipocytes can be formed from progenitor cells. Adipocytes can be formed from preadipocytes or stem cells, such as mesenchymal stem cells. In various embodiments, an adipose or adipose-like cell differentiates from a progenitor cell.

Adipose or adipose-like cells, or tissue containing such, can be identified by detecting an adipose-specific marker (see e.g., Poulous et al. 2010 Exp Biol Med 235, 1185-1193). For example, adipose or adipose-like cells, or tissue containing such, can be identified by detecting one or more early adipose-specific markers such as ADFP (adipose differentiation related protein, aka adipophilin), pOb24, lipoprotein lipase, or pGH3. As another example, adipose or adipose-like cells, or tissue containing such, can be identified by detecting one or more later adipose-specific markers such as lipogenic enzymes (including glycerophosphate dehydrogenases generally and glycerol-3-phosphate dehydrogenase specifically), aP2, and adipsin. As another example, adipose or adipose-like cells, or tissue containing such, can be identified by detecting adipose stem cells via the CD34 marker. As another example, adipose or adipose-like cells, or tissue containing such, can be identified by detecting accumulation of tri-acyl glycerol. As another example, adipose or adipose-like cells, or tissue containing such, can be identified by detecting lipid accumulation using Oil red-O (see Example 1). An adipose-like cell can be a cell that displays one or more adipose-cell related markers, such as any of those adipose markers described above.

In some embodiments, adipose or adipose-like cells can be formed in a scaffold or matrix material at a density of about 0.0001 million cells (M) ml⁻¹ to about 1000 M ml⁻¹. For example, adipose or adipose-like cells can be formed in a scaffold or matrix material at a density of about 1 M ml⁻¹, 5 M ml⁻¹, 10 M ml⁻¹, 15 M ml⁻¹, 20 M ml⁻¹, 25 M ml⁻¹, 30 M ml⁻¹, 35 M ml⁻¹, 40 M ml⁻¹, 45 M ml⁻¹, 50 M ml⁻¹, 55 M ml⁻¹, 60 M ml⁻¹, 65 M ml⁻¹, 70 M ml⁻¹, 75 M ml⁻¹, 80 M ml⁻¹, 85 M ml⁻¹, 90 M ml⁻¹, 95 M ml⁻¹, or 100 M ml⁻¹.

Attenuators of Adipogenic Inhibition

The present disclosure provides compositions and methods for reducing or eliminating inhibition of adipogenesis. In various embodiments, a protein kinase agonist or inhibitor, such as an epidermal growth factor receptor (EGFR) antagonist can be used to increase adipogenesis.

A secretase γ (gamma) inhibitor can be used in compositions and methods described herein so as to increase adipogenesis. Secretase γ is an integral membrane protein that cleaves single-pass transmembrane proteins. Secretase γ inhibitors are commercially available from a variety of sources (e.g., Tocris Bioscience, MO; Santa Cruz Biotechnology, Inc., CA; Axon Medchem, The Netherlands). Secretase γ inhibitors include but are not limited to DAPT, JLK6, Compound W, Compound E sc-222308, and DBZ.

As shown herein, a secretase γ inhibitor resulted in up to ten-fold increases in adipogenic specific markers after three days; and after four weeks, resulted in increased levels of glycerol and leptin (see e.g., Example 2). Thus, adipogenesis can be enhanced by attenuating effects of EGF receptors, which are abundant in progenitor cells such as hematopeotic stem cells. Further, as shown herein, a Notch gamma Secretase Inhibitor (Inh1) up regulated PPARγ expression (see e.g., Example 3); up regulated C/EBPα expression (see e.g., Example 4); induced lipid accumulation (see e.g., Example 5); or initiated secretion of Adiponectin cytokine (see e.g., Example 6).

A mitogen-activated protein kinase (MAPk) inhibitor can be used in compositions and methods described herein so as to increase adipogenesis. As shown herein, a MAPk Inhibitor (Inh2) up regulated PPARγ expression (see e.g., Example 3); up regulated C/EBPα expression (see e.g., Example 4); induced lipid accumulation (see e.g., Example 5); or initiated secretion of Adiponectin cytokine (see e.g., Example 6).

An agent to attenuate adipogenic inhibitors described above (including but not limited to a secretase gamma inhibitor, a Notch gamma Secretase Inhibitor, or a MAPk inhibitor) can be included in the scaffold or matrix material. An agent to attenuate adipogenic inhibitors described above (including but not limited to a secretase gamma inhibitor, a Notch gamma Secretase Inhibitor, or a MAPk inhibitor) can be included in the cell homing composition. An agent to attenuate adipogenic inhibitors described above (including but not limited to a secretase gamma inhibitor, a Notch gamma Secretase Inhibitor, or a MAPk inhibitor) can be included in the adipogenic composition.

An agent to attenuate adipogenic inhibitors (including but not limited to a secretase gamma inhibitor, a Notch gamma Secretase Inhibitor, or a MAPk inhibitor) can be present at a concentration of about 0.1 μM to about 1,000 μM.

For example, a secretase γ inhibitor can be present at a concentration of about 1.0 μM to about 100 μM. As another example, a secretase γ inhibitor can be present at a concentration of about 0.1, about 0.5, about 1, about 2, about 3, about 4, about 5, about 6, about 7, about 8, about 9, about 10, about 11, about 12, about 13, about 14, about 15, about 16, about 17, about 18, about 19, or about 20 μM, about 50 μM, or about 100 μM. As another example, a secretase γ inhibitor can be present at a concentration of about 10 μM (see Example 2). A

As another example, a Notch gamma Secretase Inhibitor can be present at a concentration of about 1.0 μM to about 100 μM. As another example, a Notch gamma Secretase Inhibitor can be present at a concentration of about 0.1, about 0.5, about 1, about 2, about 3, about 4, about 5, about 6, about 7, about 8, about 9, about 10, about 11, about 12, about 13, about 14, about 15, about 16, about 17, about 18, about 19, or about 20 μM, about 50 μM, or about 100 μM. As another example, a Notch gamma Secretase Inhibitor can be present at a concentration of about 10 μM.

As another example, a MAPk inhibitor can be present at a concentration of about 1.0 μM to about 100 μM. As another example, a MAPk inhibitor can be present at a concentration of about 0.1, about 0.5, about 1, about 2, about 3, about 4, about 5, about 6, about 7, about 8, about 9, about 10, about 11, about 12, about 13, about 14, about 15, about 16, about 17, about 18, about 19, or about 20 μM, about 50 μM, or about 100 μM. As another example, a MAPk inhibitor can be present at a concentration of about 10 μM.

In some embodiments, an agent to attenuate adipogenic inhibitors (including but not limited to a secretase gamma inhibitor, a Notch gamma Secretase Inhibitor, or a MAPk inhibitor) can be encapsulated in a microsphere.

For example, a secretase γ inhibitor can be encapsulated in a microsphere at a ratio of about 0.1/250 to about 250/250 (μg inhibitor per mg microsphere material). For example, a secretase γ inhibitor can be encapsulated in a microsphere at a ratio of about 0.1/250; about 0.5/250; about 1/250; about 2/250; about 3/250; about 4/250; about 5/250; about 6/250; about 7/250; about 8/250; about 9/250; about 10/250; about 11/250; about 12/250; about 13/250; about 14/250; about 15/250; about 16/250; about 17/250; about 18/250; about 19/250; about 20/250; about 25/250; about 30/250; about 35/250; about 40/250; about 45/250; about 50/250; about 60/250; about 70/250; about 80/250; about 90/250; about 100/250; about 150/250; about 200/250; or about 250/250 μg inhibitor per mg microsphere material.

As another example, a Notch gamma Secretase Inhibitor can be encapsulated in a microsphere at a ratio of about 0.1/250 to about 250/250 (μg inhibitor per mg microsphere material). For example, Notch gamma Secretase Inhibitor can be encapsulated in a microsphere at a ratio of about 0.1/250; about 0.5/250; about 1/250; about 2/250; about 3/250; about 4/250; about 5/250; about 6/250; about 7/250; about 8/250; about 9/250; about 10/250; about 11/250; about 12/250; about 13/250; about 14/250; about 15/250; about 16/250; about 17/250; about 18/250; about 19/250; about 20/250; about 25/250; about 30/250; about 35/250; about 40/250; about 45/250; about 50/250; about 60/250; about 70/250; about 80/250; about 90/250; about 100/250; about 150/250; about 200/250; or about 250/250 μg inhibitor per mg microsphere material.

As another example, a MAPk inhibitor can be encapsulated in a microsphere at a ratio of about 0.1/250 to about 250/250 (μg inhibitor per mg microsphere material). For example, a MAPk inhibitor can be encapsulated in a microsphere at a ratio of about 0.1/250; about 0.5/250; about 1/250; about 2/250; about 3/250; about 4/250; about 5/250; about 6/250; about 7/250; about 8/250; about 9/250; about 10/250; about 11/250; about 12/250; about 13/250; about 14/250; about 15/250; about 16/250; about 17/250; about 18/250; about 19/250; about 20/250; about 25/250; about 30/250; about 35/250; about 40/250; about 45/250; about 50/250; about 60/250; about 70/250; about 80/250; about 90/250; about 100/250; about 150/250; about 200/250; or about 250/250 μg inhibitor per mg microsphere material.

In some embodiments, one or more agents to attenuate adipogenic inhibitors (including but not limited to a secretase gamma inhibitor, a Notch gamma Secretase Inhibitor, or a MAPk inhibitor) can be used sequentially or concurrently in or with compositions or methods described herein. For example, combined treatment of a Notch gamma Secretase Inhibitor and a MAPk inhibitor was shown to provide additive or synergistic results in up regulating PPARγ expression (see e.g., Example 3); up regulating C/EBPα expression (see e.g., Example 4); inducing lipid accumulation (see e.g., Example 5); or initiating secretion of Adiponectin cytokine (see e.g., Example 6).

Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, preferably in purified form, together with a suitable amount of carrier so as to provide the form for proper administration to the subject.

The formulation should suit the mode of administration. The agents of use with the current invention can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Microspheres

Various embodiments described herein employ controlled release compositions. For example, a controlled release adipogenic composition can be introduced into a scaffold or matrix material. As another example, a controlled release cell homing composition can be introduced into a scaffold or matrix material. The controlled release systems described herein can allow for controlled release of separate chemicals or compositions at similar or at different rates. For example, a controlled release system can allow the release of separate chemicals or compositions at different rates, so as to provide, e.g., a cell homing composition at a different rate, including faster or slower, than an adipogenic composition. As another example, a controlled release system as described herein can provide for the delivery of one compound or composition sooner than a second compound or composition. As a specific example, a controlled release system described herein can release a portion or a substantial portion of the cell homing composition earlier than the adipogenic composition. For example, the cell homing composition can be released about 1, about 2, about 3, about 4, about 5, about 6, about 7, about 8, about 9, or about 10 or more days before the adipogenic composition. As another specific example, a controlled release system described herein can release a portion or a substantial portion of an adipogenic composition earlier than a cell homing composition. For example, the adipogenic composition can be released about 1, about 2, about 3, about 4, about 5, about 6, about 7, about 8, about 9, or about 10 or more days before the cell homing composition.

Compositions described herein (e.g., adipogenic composition, cell homing composition) can be introduced into or onto a scaffold or matrix material via a carrier based system, such as an encapsulation vehicle. For example, an adipogenic composition or a cell homing composition can be encapsulated within a polymeric delivery systems so as to provide for controlled release of such compositions from within the scaffold or matrix material. Such vehicles are useful as slow release compositions. For example, various compositions can be micro-encapsulated to provide for enhanced stability or prolonged delivery. Encapsulation vehicles include, but are not limited to, microparticles, liposomes, microspheres, or the like, or a combination of any of the above to provide the desired release profile in varying proportions. Other methods of controlled-release delivery of agents will be known to the skilled artisan. Moreover, these and other systems can be combined or modified to optimize the integration/release of agents within the scaffold or matrix material.

For example, the polymeric delivery system can be a polymeric microsphere, preferably a PLGA polymeric microspheres. A variety of polymeric delivery systems, as well as methods for encapsulating a molecule such as a growth factor, are known to the art (see e.g., Varde and Pack (2004) Expert Opin Biol Ther 4, 35-51). Polymeric microspheres can be produced using naturally occurring or synthetic polymers and are particulate systems in the size range of 0.1 to 500 μm. Polymeric micelles and polymeromes are polymeric delivery vehicles with similar characteristics to microspheres and can also facilitate encapsulation and matrix integration of the compounds described herein. Fabrication, encapsulation, and stabilization of microspheres for a variety of payloads are within the skill of the art (see e.g., Varde & Pack (2004) Expert Opin. Biol. 4(1) 35-51). The release rate of the microspheres can be tailored by type of polymer, polymer molecular weight, copolymer composition, excipients added to the microsphere formulation, and microsphere size. Polymer materials useful for forming microspheres include PLA, PLGA, PLGA coated with DPPC, DPPC, DSPC, EVAc, gelatin, albumin, chitosan, dextran, DL-PLG, SDLMs, PEG (e.g., ProMaxx), sodium hyaluronate, diketopiperazine derivatives (e.g., Technosphere), calcium phosphate-PEG particles, and/or oligosaccharide derivative DPPG (e.g., Solidose). Encapsulation can be accomplished, for example, using a water/oil single emulsion method, a water-oil-water double emulsion method, or lyophilization. Several commercial encapsulation technologies are available (e.g., ProLease®, Alkerme).

Liposomes can also be used to integrate compositions described herein with a scaffold or matrix material. The agent carrying capacity and release rate of liposomes can depend on the lipid composition, size, charge, drug/lipid ratio, and method of delivery. Conventional liposomes are composed of neutral or anionic lipids (natural or synthetic). Commonly used lipids are lecithins such as phosphatidylcholines, phosphatidylethanolamines, sphingomyelins, phosphatidylserines, phosphatidylglycerols, and phosphatidylinositols. Liposome encapsulation methods are commonly known in the arts (Galovic et al. (2002) Eur. J. Pharm. Sci. 15, 441-448; Wagner et al. (2002) J. Liposome Res. 12, 259-270). Targeted liposomes and reactive liposomes can also be used in combination with the agents and matrix. Targeted liposomes have targeting ligands, such as monoclonal antibodies or lectins, attached to their surface, allowing interaction with specific receptors and/or cell types. Reactive or polymorphic liposomes include a wide range of liposomes, the common property of which is their tendency to change their phase and structure upon a particular interaction (e.g., pH-sensitive liposomes) (see e.g., Lasic (1997) Liposomes in Gene Delivery, CRC Press, FL).

Scaffold

Various embodiments described herein employ a scaffold or matrix material. For example, a cell homing composition or an adipogenic composition can be included in or on a scaffold.

The scaffold optionally does not comprise a transplanted mammalian cell, i.e., no cell is applied to the scaffold; any cell present in the scaffold migrated into the scaffold.

A scaffold can be fabricated with any matrix material recognized as useful by the skilled artisan. A matrix material can be a biocompatible material that generally forms a porous, microcellular scaffold, which provides a physical support for cells migrating thereto. Such matrix materials can: allow cell attachment and migration; deliver and retain cells and biochemical factors; enable diffusion of cell nutrients and expressed products; or exert certain mechanical and biological influences to modify the behavior of the cell phase. The matrix material generally forms a porous, microcellular scaffold of a biocompatible material that provides a physical support and an adhesive substrate for recruitment and growth of cells during in vitro or in vivo culturing.

Suitable scaffold and matrix materials are discussed in, for example, Ma and Elisseeff, ed. (2005) Scaffolding In Tissue Engineering, CRC, ISBN 1574445219; Saltzman (2004) Tissue Engineering: Engineering Principles for the Design of Replacement Organs and Tissues, Oxford ISBN 019514130X. For example, matrix materials can be, at least in part, solid xenogenic (e.g., hydroxyapatite) (Kuboki et al. 1995 Connect Tissue Res 32, 219-226; Murata et al. 1998 Int J Oral Maxillofac Surg 27, 391-396), solid alloplastic (polyethylene polymers) materials (Saito and Takaoka 2003 Biomaterials 24 2287-93; Isobe et al. 1999 J Oral Maxillofac Surg 57, 695-8), or gels of autogenous (Sweeney et al. 1995. J Neurosurg 83, 710-715), allogenic (Bax et al. 1999 Calcif Tissue Int 65, 83-89; Viljanen et al. 1997 Int J Oral Maxillofac Surg 26, 389-393), or alloplastic origin (Santos et al. 1998. J Biomed Mater Res 41, 87-94), and combinations of the above (Alpaslan et al. 1996 Br J of Oral Maxillofac Surg 34, 414-418).

The matrix comprising the scaffold can have an adequate porosity and an adequate pore size so as to facilitate cell recruitment and diffusion throughout the whole structure of both cells and nutrients. The matrix can be biodegradable providing for absorption of the matrix by the surrounding tissues, which can eliminate the necessity of a surgical removal. The rate at which degradation occurs can coincide as much as possible with the rate of tissue or organ formation. Thus, while cells are fabricating their own natural structure around themselves, the matrix is able to provide structural integrity and eventually break down, leaving the neotissue, newly formed tissue or organ which can assume the mechanical load. The matrix can be an injectable matrix in some configurations. The matrix can be delivered to a tissue using minimally invasive endoscopic procedures.

The scaffold can comprise a matrix material having different phases of viscosity. For example, a matrix can have a substantially liquid phase or a substantially gelled phase. The transition between phases can be stimulated by a variety of factors including, but limited to, light, chemical, magnetic, electrical, and mechanical stimulus. For example, the matrix can be a thermosensitive matrix with a substantially liquid phase at about room temperature and a substantially gelled phase at about body temperature. The liquid phase of the matrix can have a lower viscosity that provides for optimal distribution of growth factors or other additives and injectability, while the solid phase of the matrix can have an elevated viscosity that provides for matrix retention at or within the target tissue.

The scaffold can comprise a matrix material formed of synthetic polymers. Such synthetic polymers include, but are not limited to, polyurethanes, polyorthoesters, polyvinyl alcohol, polyamides, polycarbonates, polyvinyl pyrrolidone, marine adhesive proteins, cyanoacrylates, analogs, mixtures, combinations and derivatives of the above. Alternatively, the matrix can be formed of naturally occurring biopolymers. Such naturally occurring biopolymers include, but are not limited to, fibrin, fibrinogen, fibronectin, collagen, and other suitable biopolymers. Also, the matrix can be formed from a mixture of naturally occurring biopolymers and synthetic polymers.

The scaffold can include one or more matrix materials including, but not limited to, a collagen gel, a polyvinyl alcohol sponge, a poly(D,L-lactide-co-glycolide) fiber matrix, a polyglactin fiber, a calcium alginate gel, a polyglycolic acid mesh, polyester (e.g., poly-(L-lactic acid) or a polyanhydride), a polysaccharide (e.g. alginate), polyphosphazene, polyacrylate, or a polyethylene oxide-polypropylene glycol block copolymer. Matrices can be produced from proteins (e.g. extracellular matrix proteins such as fibrin, collagen, and fibronectin), polymers (e.g., polyvinylpyrrolidone), or hyaluronic acid. Synthetic polymers can also be used, including bioerodible polymers (e.g., poly(lactide), poly(glycolic acid), poly(lactide-co-glycolide), poly(caprolactone), polycarbonates, polyamides, polyanhydrides, polyamino acids, polyortho esters, polyacetals, polycyanoacrylates), degradable polyurethanes, non-erodible polymers (e.g., polyacrylates, ethylene-vinyl acetate polymers and other acyl substituted cellulose acetates and derivatives thereof), non-erodible polyurethanes, polystyrenes, polyvinyl chloride, polyvinyl fluoride, poly(vinylimidazole), chlorosulphonated polyolifins, polyethylene oxide, polyvinyl alcohol, Teflon®, or nylon.

The scaffold can further comprise any other bioactive molecule, for example an antibiotic or an additional chemotactic growth factor or another osteogenic, dentinogenic, amelogenic, or cementogenic growth factor. In some embodiments, the scaffold is strengthened, through the addition of, e.g., human serum albumin (HSA), hydroxyethyl starch, dextran, or combinations thereof. Suitable concentrations of these compounds for use in the compositions of the application are known to those of skill in the art, or can be readily ascertained without undue experimentation.

The concentration of a compound or a composition in the scaffold will vary with the nature of the compound or composition, its physiological role, and desired therapeutic or diagnostic effect. A therapeutically effective amount is generally a sufficient concentration of therapeutic agent to display the desired effect without undue toxicity. For example, the matrix can include an adipogenic composition at the above described concentrations. As another example, the matrix can include an cell homing composition at the above described concentrations. The compound can be incorporated into the scaffold or matrix material by any known method. In some embodiments, the compound is imbedded in a gel, e.g., a collagen gel incorporated into the pores of the scaffold or matrix material.

Alternatively, chemical modification methods can be used to covalently link a compound or a composition to a matrix material. The surface functional groups of the matrix can be coupled with reactive functional groups of a compound or a composition to form covalent bonds using coupling agents well known in the art such as aldehyde compounds, carbodiimides, and the like. Additionally, a spacer molecule can be used to gap the surface reactive groups and the reactive groups of the biomolecules to allow more flexibility of such molecules on the surface of the matrix. Other similar methods of attaching biomolecules to the interior or exterior of a matrix will be known to one of skill in the art.

Pores and channels of the scaffold can be engineered to be of various diameters. For example, the pores of the scaffold can have a diameter range from micrometers to millimeters. In some embodiments, the pores of the matrix material include microchannels. Microchannels generally have an average diameter of about 0.1 μm to about 1,000 μm, e.g., about 50 μm to about 500 μm (for example about 100 μm, 150 μm, about 200 μm, about 250 μm, about 300 μm, about 350 μm, about 400 μm, about 450 μm, about 500 μm, or about 550 μm). One skilled in the art will understand that the distribution of microchannel diameters can have any distribution including a normal distribution or a non-normal distribution. In some embodiments, microchannels are a naturally occurring feature of the matrix material(s). In other embodiments, microchannels are engineered to occur in the matrix materials.

Several methods can be used for fabrication of porous scaffolds, including particulate leaching, gas foaming, electrospinning, freeze drying, foaming of ceramic from slurry, and the formation of polymeric sponge (see e.g., Example 1). Other methods can be used for fabrication of porous scaffolds include computer aided design (CAD) and synthesizing the scaffold with a bioplotter (e.g., solid freeform fabrication) (e.g., Bioplotter™, EnvisionTec, Germany).

Biologic drugs that can be added to compositions of the invention include immunomodulators and other biological response modifiers. A biological response modifier generally encompasses a biomolecule (e.g., peptide, peptide fragment, polysaccharide, lipid, antibody) that is involved in modifying a biological response, such as the immune response or tissue or organ growth and repair, in a manner that enhances a particular desired therapeutic effect, for example, the cytolysis of bacterial cells or the growth of tissue- or organ-specific cells or vascularization. Biologic drugs can also be incorporated directly into the matrix component. Those of skill in the art will know, or can readily ascertain, other substances which can act as suitable non-biologic and biologic drugs.

Compositions described herein can also be modified to incorporate a diagnostic agent, such as a radiopaque agent. The presence of such agents can allow the physician to monitor the progression of wound healing occurring internally. Such compounds include barium sulfate as well as various organic compounds containing iodine. Examples of these latter compounds include iocetamic acid, iodipamide, iodoxamate meglumine, iopanoic acid, as well as diatrizoate derivatives, such as diatrizoate sodium. Other contrast agents that can be utilized in the compositions can be readily ascertained by those of skill in the art and can include, for example, the use of radiolabeled fatty acids or analogs thereof.

The concentration of an agent in the composition will vary with the nature of the compound, its physiological role, and desired therapeutic or diagnostic effect. A therapeutically effective amount is generally a sufficient concentration of therapeutic agent to display the desired effect without undue toxicity. A diagnostically effective amount is generally a concentration of diagnostic agent which is effective in allowing the monitoring of the integration of the tissue graft, while minimizing potential toxicity. In any event, the desired concentration in a particular instance for a particular compound is readily ascertainable by one of skill in the art.

Implanting

Various embodiments provide compositions and methods to recruit, home, or induce differentiation of progenitor cells by using a cell homing composition and subsequently promote or induce differentiation of recruited progenitor cells to form adipose or adipose-like cells using an adipogenic composition. A cell homing composition, an adipogenic composition, and a scaffold or matrix can be implanted in a subject so as to recruit endogenous progenitor cells into the scaffold or matrix material and differentiate recruited progenitor cells to adipose or adipose-like cells.

In some embodiments, methods of causing progenitor cells to migrate to a scaffold and differentiate to form adipose or adipose-like cells in the scaffold are provided. The method can include placing a scaffold containing a cell homing composition and an adipogenic composition in fluid communication with cells. As used herein, a scaffold is in “fluid communication” with a cell if the cell has no physical barrier (e.g., a basement membrane, areolar connective tissue, adipose connective tissue, etc.) preventing the cell from migrating to the scaffold. Without being bound to any particular mechanism, it is believed that the cell migrates to the scaffold along a moist path from its source, in response to the presence of a cell homing composition forming a concentration gradient to the cell, and thereby influencing the cell to migrate toward the higher concentrations of the cell homing composition in the scaffold.

The scaffold optionally does not comprise a transplanted mammalian cell, i.e., no cell is applied to the scaffold; any cell present in the scaffold migrated into the scaffold. A scaffold is generally understood to be a three-dimensional structure into which cells, tissue, vessels, etc., can grow, colonize and populate when the scaffold is placed into a tissue site. A scaffold of the method can be as discussed herein.

The compositions and methods described herein hold significant clinical value because of their ability to be recruit endogenous progenitor cells, thereby optionally avoiding transplant of cells to a subject. A determination of the need for treatment will typically be assessed by a history and physical exam consistent with the tissue or organ defect at issue. A subject in need of the therapeutic methods and compositions described herein can be a subject having, diagnosed with, suspected of having, or at risk for developing a tissue or organ defect, such as a soft tissue defect. A soft tissue defect is generally understood as a void within the subcutaneous fat layer of the skin that often results in a change in the “normal” tissue contour. Soft tissue defects include, but are not limited to, traumatic injury (e.g., significant burns), tumor resections (e.g., mastectomy and carcinoma removal), and congenital defects.

Diagnosis of the various conditions treatable by the methods described herein is within the skill of the art. The subject can be an animal subject, including, but not limited to, mammals, reptiles, and avians, more preferably horses, cows, dogs, cats, sheep, pigs, mice, rats, monkeys, guinea pigs, and chickens, and most preferably a human.

An effective amount of a cell homing composition can be that which can induce recruitment of progenitor cells or migration of progenitor cells. An effective amount of an adipogenic composition can be that which can induce differentiation of progenitor cells to adipose or adipose-like cells. An effective amount of a scaffold or matrix material containing cell homing composition and an adipogenic composition can be that which can induce recruitment of progenitor cells or migration of progenitor cells and induce differentiation of recruited progenitor cells to adipose or adipose-like cells. An effective amount of a scaffold or matrix material containing a cell homing composition and an adipogenic composition can be that which can recruit and induce migration of a sufficient number of progenitor cells and induce at least a portion of recruited progenitor cells to form adipose or adipose-like cells so as to increase biological function of a tissue or organ. An effective amount of a scaffold or matrix material containing cell homing composition and an adipogenic composition can be that which restores function or appearance to soft tissue.

As an example, a subject in need can have a adipose cell or tissue deficiency of at least about 5%, about 10%, about 25%, about 50%, about 75%, about 90% or more, and compositions and methods described herein can provide an increase in number or function of adipose cells or tissues. As another example, a subject in need can have damage to a tissue or organ, and the method can provide an increase in biological function of the tissue or organ by at least about 5%, about 10%, about 25%, about 50%, about 75%, about 90%, about 100%, or about 200%, or even by as much as about 300%, about 400%, or about 500%. As yet another example, the subject in need can have an adipose-related disease, disorder, or condition, and the method provides an engineered scaffold sufficient that can recruit progenitor cells and form adipose cells or tissue sufficient to ameliorate or stabilize the disease, disorder, or condition. For example, the subject can have a disease, disorder, or condition that results in the loss, atrophy, dysfunction, and/or death of adipose cells. In a further example, the subject in need can have an increased risk of developing a disease, disorder, or condition that is delayed or prevented by the method. As yet another example, the subject in need can have experienced death or dysfunction of adipose cells as the result of a side effect of a medication used for the treatment of another disease or disorder, for example from the use of Copaxone (glatiramer acetate) as a treatment for multiple sclerosis; or from the use of anti-retroviral therapy in HIV-positive individuals.

The tissue or organ can be selected from adipose, bladder, brain, nervous tissue, glia, esophagus, fallopian tube, heart, pancreas, intestines, gall bladder, kidney, liver, lung, ovaries, prostate, spinal cord, spleen, stomach, testes, thymus, thyroid, trachea, urogenital tract, ureter, urethra, uterus, breast, skeletal muscle, skin, tooth, bone, and cartilage. Progenitor cells can be from the same subject into which the scaffold and/or matrix is grafted. Alternatively, the progenitor cells can be from the same species, or even different species.

Implantation of an engineered construct is within the skill of the art. The scaffold or matrix material can be either fully or partially implanted into a tissue or organ of the subject to become a functioning part thereof. Preferably, the implant initially attaches to and communicates with the host through a cellular monolayer. Over time, endogenous cells can migrate into the scaffold to form tissue. The cells surrounding the engineered tissue can be attracted by biologically active materials, including biological response modifiers, such as polysaccharides, proteins, peptides, genes, antigens, and antibodies, which can be selectively incorporated into the matrix to provide the needed selectivity, for example, to tether the cell receptors to the matrix, stimulate cell migration into the matrix, or both. The matrix can comprise a gelled phase and interconnecting channels that allow for cell migration, augmented by both biological and physical-chemical gradients. For example, cells surrounding the implanted matrix can be attracted by biologically active materials including IGF1 and bFGF. One of skill in the art will recognize and know how to use other biologically active materials that are appropriate for attracting cells to the matrix.

The methods, compositions, and devices described herein can include concurrent or sequential treatment with one or more of enzymes, ions, growth factors, and biologic agents, such as thrombin and calcium, or combinations thereof. The methods, compositions, and devices described herein can include concurrent or sequential treatment with non-biologic and/or biologic drugs.

When used in the treatments described herein, a therapeutically effective amount of an adipogenic composition or a cell homing composition can be employed in pure form or, where such forms exist, in pharmaceutically acceptable salt form and with or without a pharmaceutically acceptable excipient. For example, the compounds described herein can be administered, at a reasonable benefit/risk ratio applicable to any medical treatment, in a sufficient amount to increase biological function of a tissue or organ.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

The specific therapeutically effective dose level for any particular subject will depend upon a variety of factors including the disorder being treated and the severity of the disorder; the location and size of the site of treatment; activity of the specific compound employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present invention will be decided by an attending physician within the scope of sound medical judgment.

Administration of compositions or scaffold comprising compositions described herein can occur as a single event or over a time course of treatment. For example, administration can be daily, weekly, bi-weekly, or monthly.

Treatment in accord with the methods described herein can be performed prior to, concurrent with, or after conventional treatment modalities for a tissue or organ defect. Compositions or scaffold comprising compositions described herein can be administered simultaneously or sequentially with another agent, such as an antibiotic, an antiinflammatory, or another agent. For example, a administration can occur simultaneously with another agent, such as an antibiotic or an anti-inflammatory.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a scaffold, matrix materials, a cell homing composition, an adipogenic composition, and controlled release systems, such as microspheres, optionally encapsulating other components. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present invention and to guide those of ordinary skill in the art in the practice of the present invention. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments described herein are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments described herein are approximations, the numerical values set forth in the specific examples are reported as precisely as practicable. The numerical values presented in some embodiments described herein may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope described herein otherwise claimed. No language in the specification should be construed as indicating any non-claimed element essential to the practice of the present disclosure.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present invention.

Having described the invention in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

This example shows homing of cells into a scaffold and adipogenesis in vitro and in vivo.

Porous poly(lactic-co-glycolic acid) (PLGA) scaffolds were fabricated. Two grams of 85:15 PLGA were dissolved in 30 ml of di-chloro-methane (DCM). To generate porous scaffolds, a salt-leaching method was used. NaCl crystals were sieved to generate crystals ranging from 130 μm to 600 μm. The PLGA-solution was gently poured over 18 grams of sieved NaCl crystals, and the DCM was allowed to evaporate overnight in a fume hood. The next day, five μm diameter round disks were punched from the PLGA. The disks were placed in distilled water for 48 hours, the water replaced every hour the first eight hours, then twice a day the remaining time. The scaffolds were then freeze-dried for 48 hours to remove remaining solvent and stored at −20° C. Before use, the scaffolds were sterilized in 70% ethanol for 30 minutes, then washed in PBS for 2*30 minutes and then soaked in BME medium for two hours.

Two types of microspheres were fabricated, one with the insulin-like growth factor 1 (IGF1) encapsulated, the other with supplements from adipogenic inducing medium encapsulated, denoted adipogenic microspheres. For both types, 250 mg of 50:50 PLGA was dissolved in one ml of DCM. Then, either 10 g of IGF-1 or a combination of 5.15 mg indomethacin, 1 mg insulin, 11.1 mg 3-isobutyl-1-methylxanthine and 39.2 g dexamethasone was added to the PLGA solution. The microspheres were freeze-dried for 48 hours to remove the solvent and stored at −20° C. Before use, the microspheres were sterilized using ethylene oxide.

The mouse mesenchymal cell line C3H10T1/2 (ATCC, Manassas, Va.) was expanded in BME medium. The cells were double transfected with green fluorescence protein and red fluorescent protein (Invitrogen, Carlsbad, Calif.). When enough cells were obtained, the cells were harvested and resuspended in BD puramatrix hydrogel solution (BD Biosciences, San Jose, Calif.). PLGA microspheres were added to the suspension and the solution was added to the PLGA scaffold and allowed to solidify.

A total of six groups were designed: (i) empty scaffolds; (ii) scaffolds seeded with 5×10⁵ cells of the mouse mesenchymal cell line C3H10T1/2; (iii) scaffolds with a cocktail of adipogenic factors IBMX, indometacin, dexamethasone and insulin encapsulated in microspheres; (iv) scaffolds with the adipogenic microspheres and 5×105 C3H10T1/2 cells; (v) scaffolds with insulin-like growth factor 1 (IGF1) and basic fibroblast growth factor (bFGF) encapsulated in microspheres; and (vi) scaffolds with bFGF & IGF1 microspheres and 5×105 C3H10T1/2 cells.

The scaffolds were transplanted into the abdominal fat pads of obese C57BL/6N mice for two weeks. C57BL/6NHsd were purchased from Harlan Laboratories (Indianapolis, Ind.) and fed a high caloric diet with 60% of the Kcal from fat, TD.06414 (Harlan laboratories), to generate obese mice with large fat pads. For surgery purposes, 14-15 weeks old mice were used. The mice were sedated using isoflourane, 1-5%, and a 1.5-2 cm incision was made in the lower abdominal area. A smaller incision, 0.5-1 cm, was made in the subcutaneous fat pad, and a scaffold was placed there. Six different combinations of cells and microspheres were used: empty scaffold, scaffold with 500K C3H10T1/2 cells, scaffolds with 5 mg adipogenic microspheres, scaffolds with 5 mg adipogenic microspheres and 500K C3H10T1/2 cells, scaffolds with 2.5 mg IGF-1 microspheres, scaffolds with 2.5 mg IGF-1 microspheres and 500K C3H10T1/2 cells. The scaffolds were harvested en bloc after two weeks.

Release of microencapsulated adipogenic factors from microspheres in vitro was evaluated as adipogenesis and proliferation of C3H10T1/2 cells in culture. Two weeks after transplantation in vivo, tissue grafts were harvested and sectioned.

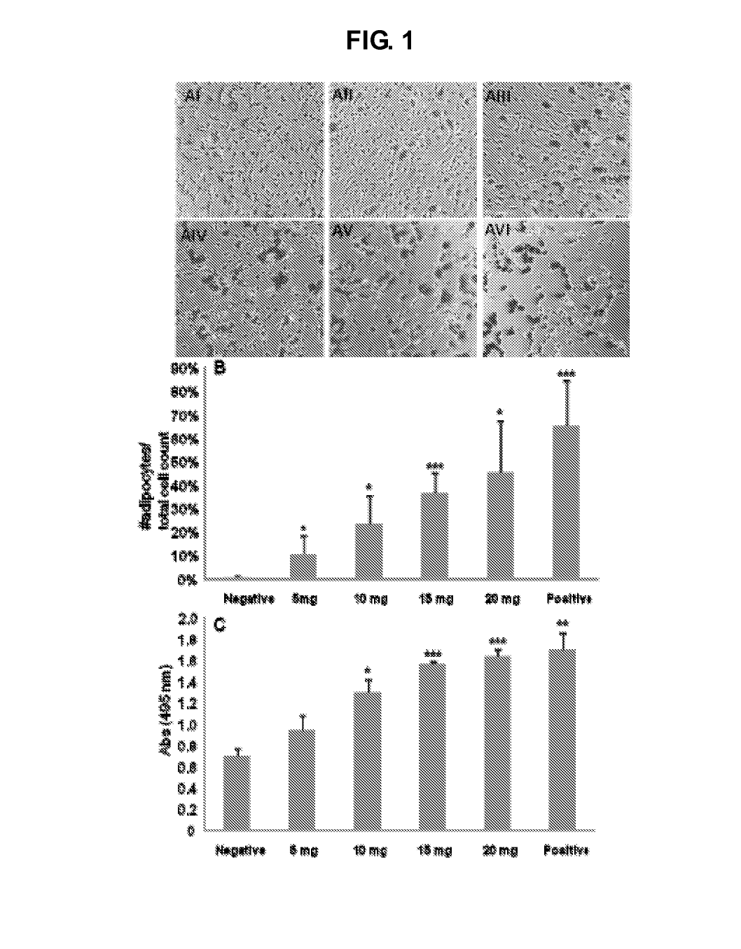

Results showed a dose dependent increase in adipogenesis (see e.g., FIG. 1).

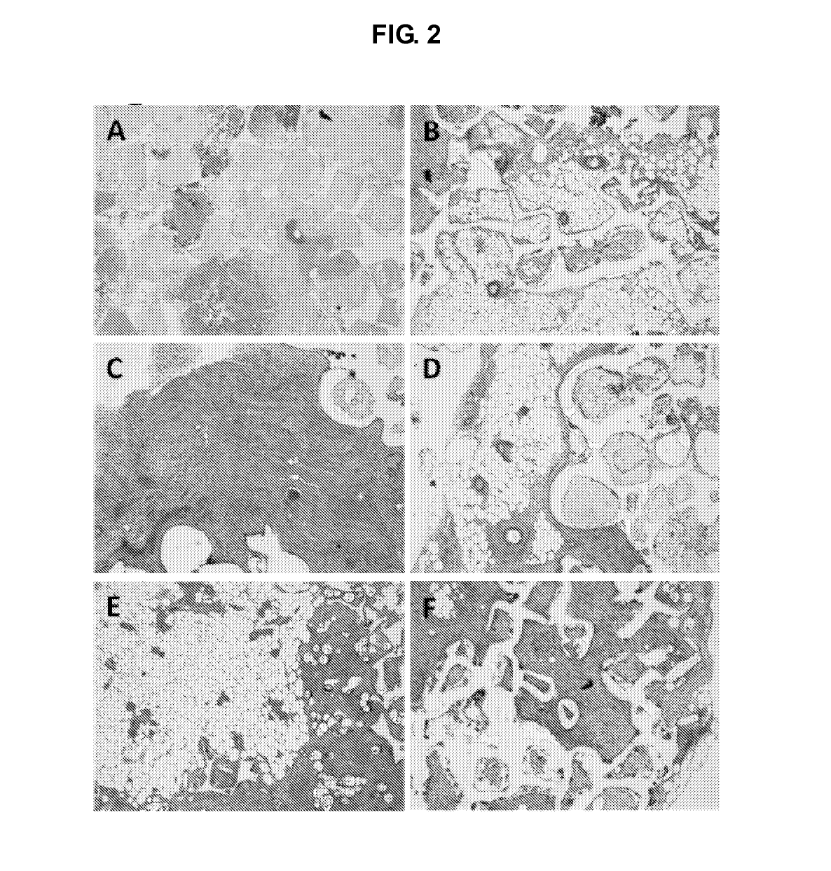

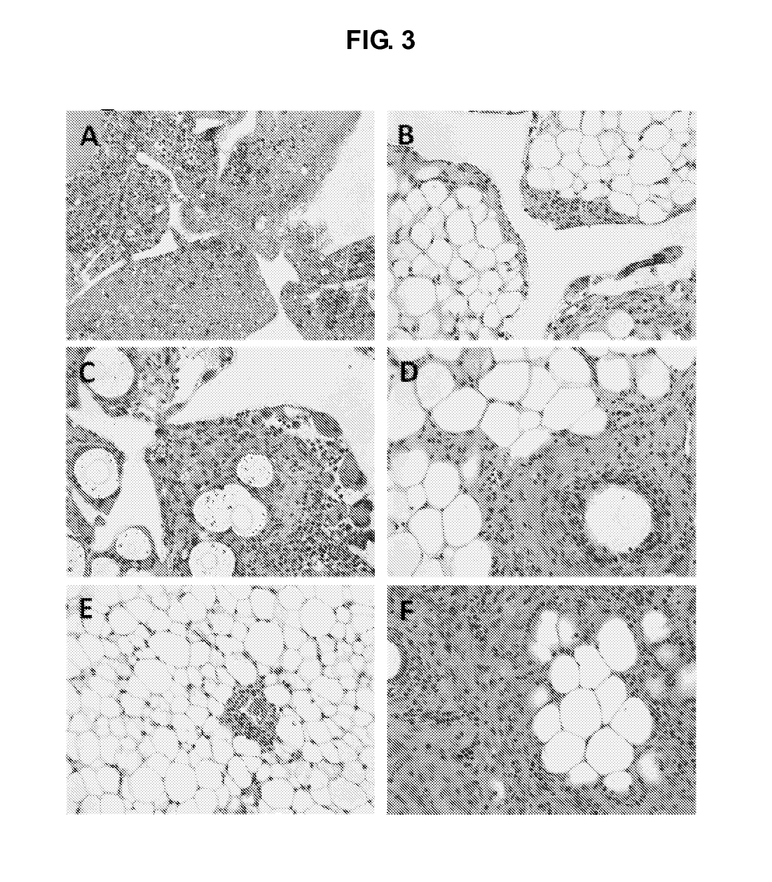

Scaffolds seeded with 5×10⁵ cells (i.e., group (ii)) and scaffolds with microencapsulated bFGF and IGF1 (i.e., group (v)) had robust adipose tissue formation (see e.g., FIG. 2B and FIG. 3B; FIG. 2D and FIG. 3D) compared to empty scaffolds (i.e., group (i)) or scaffolds supplemented with adipogenic cocktail microspheres only (i.e., group (iii)) (see e.g., FIG. 2A and FIG. 3A; FIGS. 2C and 3C). Scaffolds supplemented with bFGF and IGF1 microspheres (see e.g., FIG. 2E and FIG. 3E) had substantial adipose tissue formation in comparison to scaffolds seeded with 5×10⁵ ASCs or supplemented with bFGF and IGF1 microspheres (see e.g., FIG. 2F and FIG. 3F). Thus, controlled release of a cocktail including insulin, indometacin, IBMX and dexamethasone induces adipogenesis both in vivo and in vitro. Such a cocktail can be used to be differentiate pre-adipocytes and adipocyte stem cells homed from host tissue.

As shown above, the adipogenic cocktail microspheres promoted adipogenesis both in vivo and in vitro. And the combination of bFGF and IGF1 resulted in homing of host cells. It is expected that a combination of adipogenic cocktail microspheres and bFGF and IGF1 will result in homing of host cells and promotion of adipogenesis both in vivo and in vitro.

### Example 2

This example shows Secretase γ Inhibitor enhances adipogenesis.

Delivery of EGFR antagonists, Secretase γ Inhibitor at an optimized concentration of 10 μM within 3 days resulted in robust adipogenesis of adipocyte stem cells (ASCs), with up to 10-fold increases in the expression of adipogenic specific markers such as PPAR132, Glut4 and accelerated expression of LEPR. Quantitatively, after 4 weeks, glycerol and leptin contents of ASCs treated with EGFR antagonists were significantly higher than without EGFR antagonists. Together, the adipogenic differentiation capacity of ASCs was restored to a similar level to without HSC co-culture. Consequently, addition of endogenous EGF at a concentration of 50 ng/mL to the adipogenic medium further inhibited adipogenesis.

The results above show that adipogenesis can be enhanced by attenuating inhibitors such as EGF receptors that are abundant in hematopoietic stem cells whose co-culture was found to inhibit adipogenesis.

### Example 3

This example shows that treatment of hADSCs with a gamma secretase inhibitor is a potent up-regulator of PPARγ expression when compared to adipogenic differentiation medium (ADM).

Change in PPARγ expression of hADSCs were followed for 28 days. hADSCs were treated with: control medium; adipogenic differentiation medium (ADM); ADM plus 10 μM of Notch gamma Secretase Inhibitor (Inh1) (ADM+Inh1); ADM plus 10 μM of MAPK Inhibitor (Inh2) (ADM+Inh2); and ADM plus 10 μM of Inh1 and Inh2 (ADM+Inh1, 2).

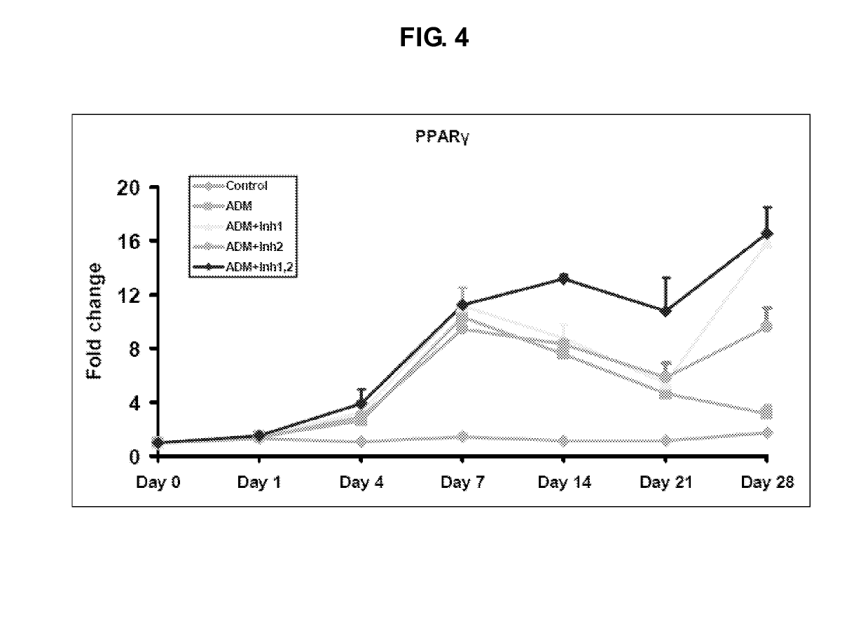

Results showed that both of the Inhibitor treatments, individually, were more potent in up regulating PPARγ expression than ADM alone. Furthermore, the combined treatment of Inh1 and Inh2 (ADM+Inh1, 2) was more potent in inducing PPARγ expression of hADSCs in vitro than the ADM and individual inhibitor 1 or 2 (see e.g., FIG. 4).

### Example 4

This example shows that treatment of hADCs with inhibitor is a potent up-regulator of C/EBPα expression when compared to ADM.

Change in C/EBPα expression of hADSCs were followed for 28 days. hADSCs were treated with: control medium; adipogenic differentiation medium (ADM); ADM plus 10 μM of Notch gamma Secretase Inhibitor (Inh1) (ADM+Inh1); ADM plus 10 μM of MAPK Inhibitor (Inh2) (ADM+Inh2); and ADM plus 10 μM of Inh1 and Inh2 (ADM+Inh1, 2).

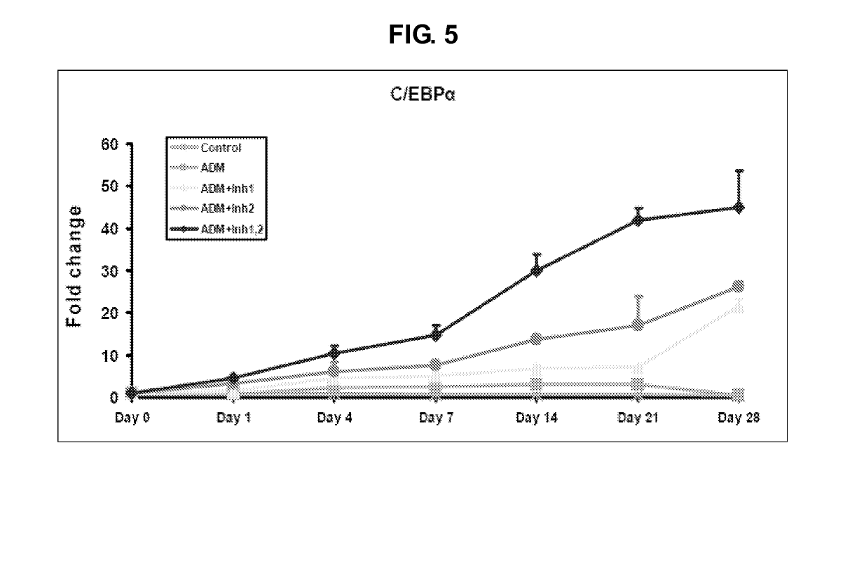

Results showed that both of the Inhibitor treatments were more potent in up regulating C/EBPα expression than ADM alone. Furthermore, the combined treatment of Inh1 and Inh2 (ADM+Inh1, 2) was more potent in inducing C/EBPα expression of hASCs in vitro than the ADM and individual inhibitor 1 or 2 (see e.g., FIG. 5)

### Example 5

This example shows that combined treatment of Inh1 and Inh2 (ADM+Inh1, 1) and Inh1 (ADM+Inh1) was more potent in inducing lipid accumulation in hASCs than ADM alone or ADM+Inh2.

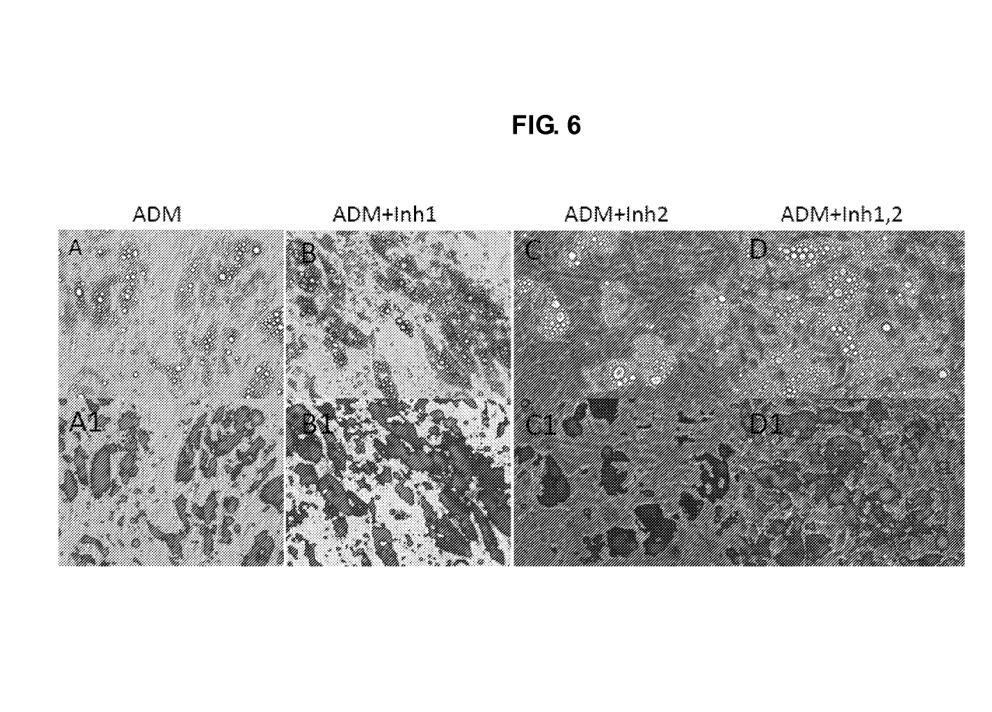

Brightfield images were produced four weeks post-treatment of hADSCs with: (A) ADM; (B) ADM+Inh1; (C) ADM+Inh2; and (D) ADM+Inh1, 2 (see e.g., FIG. 6 A-D).

Images of Lipid staining of the above hADSCs were overlayed on the Brightfield images to show that the combined treatment of Inh1 and Inh2 (ADM+Inh1, 2) and Inh1 (ADM+Inh1) were more potent in inducing lipid accumulation in hADSCs in vitro (see e.g., FIG. 6 A1-D1).

### Example 6

This example shows that the combined Inh1 and Inh2 treatment was more potent in initiating the secretion of Adiponectin cytokine in vitro.

At two and four weeks post-treatment, Adiponectin content was measure in hADSCs treated with: control medium; adipogenic differentiation medium (ADM); ADM plus 10 μM of Notch gamma Secretase Inhibitor (Inh1) (ADM+Inh1); ADM plus 10 μM of MAPK Inhibitor (Inh2) (ADM+Inh2), and ADM plus 10 μM of Inh1 and Inh2 (ADM+Inh1, 2).

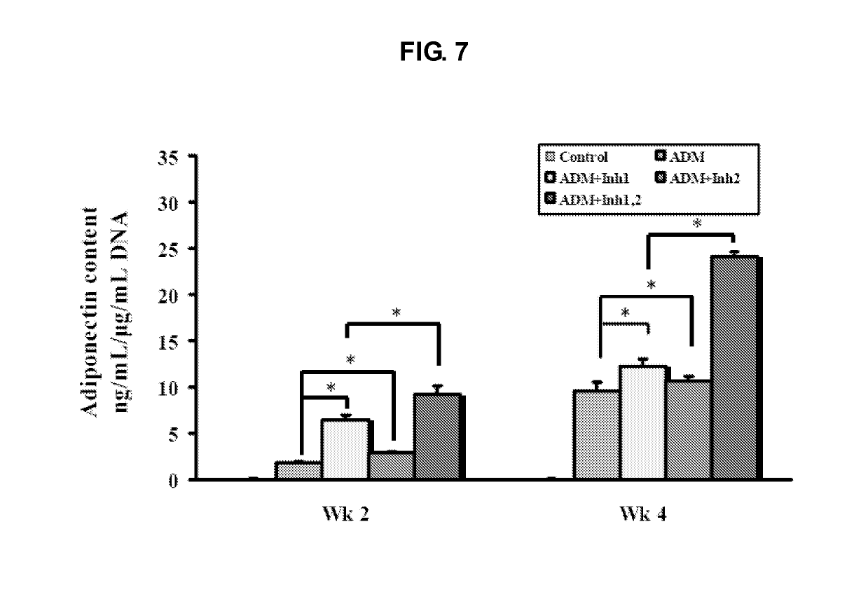

Results showed that combined treatment of Inh1 and Inh2 (ADM+Inh1, 2 group) was more potent in initiating the secretion of Adiponectin cytokine in vitro at two and four weeks (see e.g., FIG. 7).

### Example 7

This example shows that the inhibitor treatment was unsuccessful in up regulating Leptin.

At two and four weeks post-treatment, Leptin content was measured in hADSCs treated with: control medium; adipogenic differentiation medium (ADM); ADM plus 10 μM of Notch gamma Secretase Inhibitor (Inh1) (ADM+Inh1); ADM plus 10 μM of MAPK Inhibitor (Inh2) (ADM+Inh2); and ADM plus 10 μM of Inh1 and Inh2 (ADM+Inh1, 2).

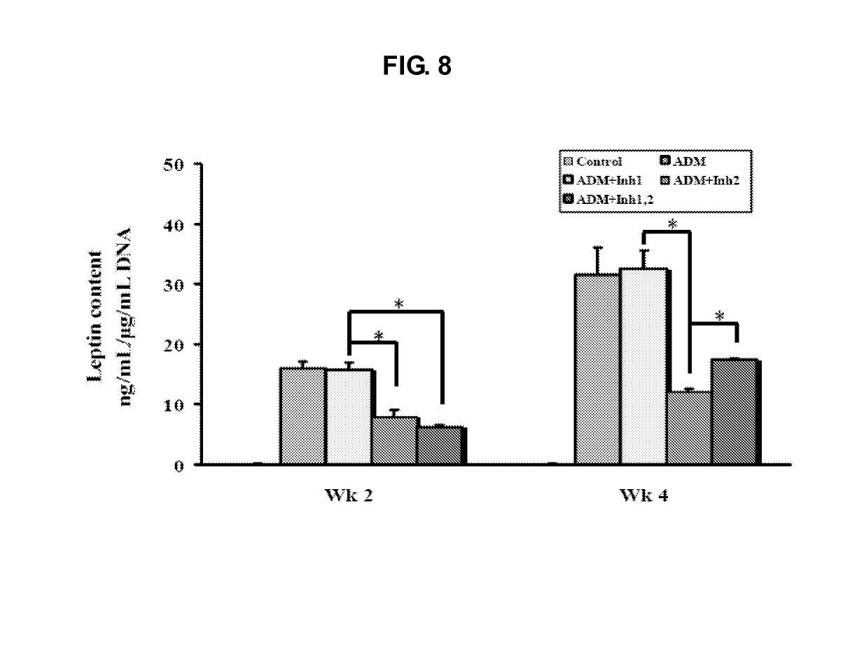

Results showed that none of the inhibitors were potent in up regulating the secretion of Leptin cytokine in vitro. Indeed, Inh2 treatment (ADM+Inh2 and ADM+Inh1, 2 group) further down regulated the secretion of Leptin cytokine in vitro (see e.g., FIG. 8).

### Example 8

This example shows that the Pyrintegrin treatment with ADM was more potent in inducing PPARγ expression in hADSCs in vitro than ADM alone.

PPARγ expression was measured for 28 days in hADSCs treated with: control medium; adipogenic differentiation medium (ADM); and ADM plus 2 μM of Pyrintegrin (ADM+Pyrintegrin).

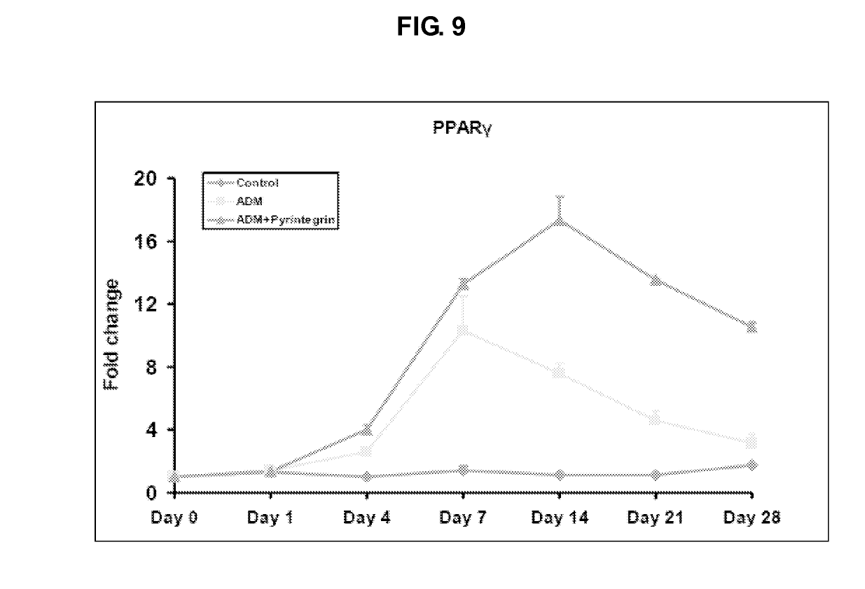

Results showed that Pyrintegrin treatment along with adipogenic medium was more potent in inducing PPARγ expression in hADSCs in vitro (see e.g., FIG. 9).

### Example 9

This example shows that Pyrintegrin treatment with ADM was more potent in inducing C/EBPα expression in hADSCs in vitro than ADM alone.

C/EBPα expression was measured for 28 days in hADSCs treated with: control medium; adipogenic differentiation medium (ADM); and ADM plus 2 μM of Pyrintegrin (ADM+Pyrintegrin).

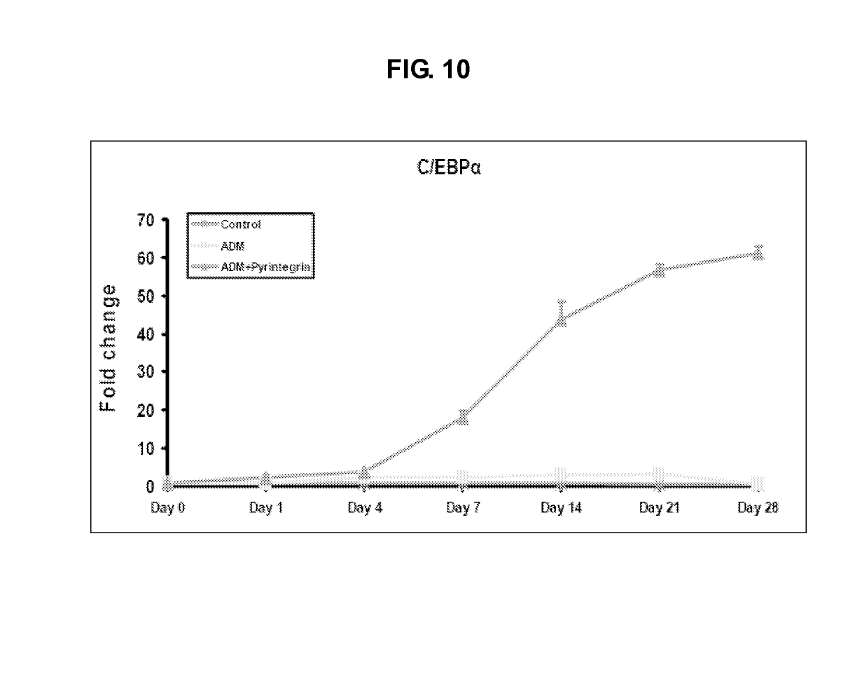

Results showed that Pyrintegrin treatment along with adipogenic medium was more potent in inducing C/EBPα expression in hADSCs in vitro (see e.g., FIG. 10).

### Example 10

This example shows that treatment of hADSCs with Pyrintegrin in addition to ADM was more potent in inducing lipid accumulation than ADM treatment alone.

Lipid staining was performed four weeks post-treatment of hADSCs with: (A) ADM; and (B) ADM plus Pyrintegrin (ADM+Pyrintegrin).

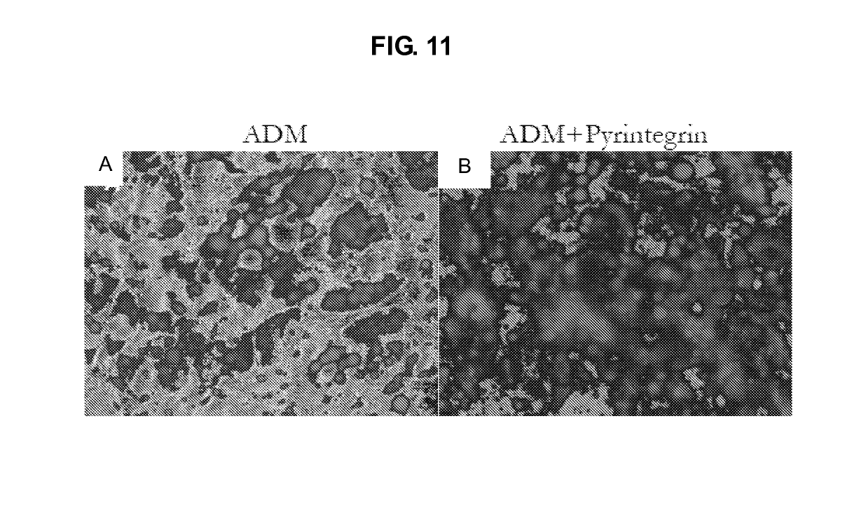

Results showed that Pyrintegrin treatment along with adipogenic medium was more potent in inducing lipid accumulation in hADSCs in vitro (see e.g., FIG. 11A-B).

### Example 11

This example shows Pyrintegrin without ADM induced adipogenic differentiation in hADSCs in vitro.

PPARγ and C/EBPα gene expression was measured at four days post-treatment in hADSCs treated with: control medium (Control); control medium plus 2 μM of Pyrintegrin (Control+Drug 2 μM); control medium plus 10 μM of Pyrintegrin (Control+Drug 10 μM) and adipogenic medium (ADM).

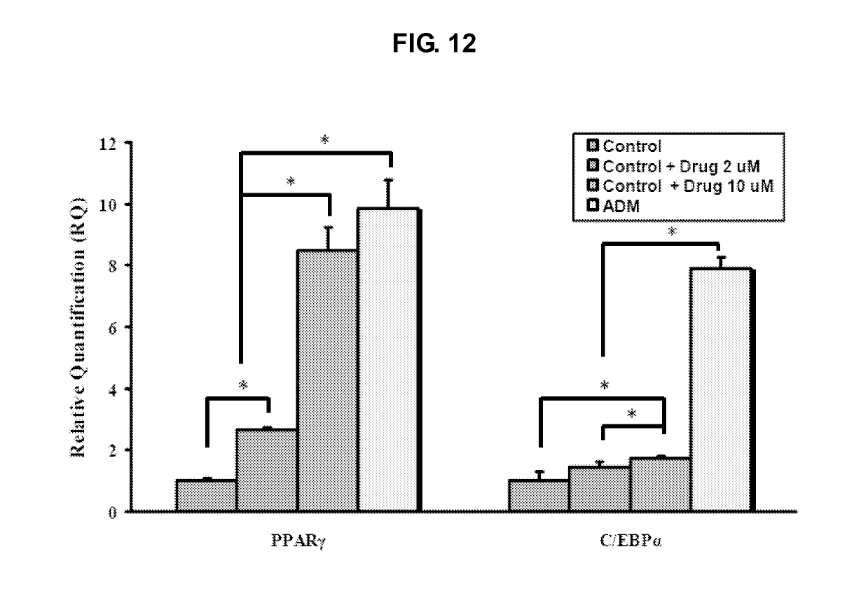

Results showed that Pyrintegrin alone can induce hADSCs towards an adipogenic differentiation pathway in vitro (see e.g., FIG. 12).

### Example 12

This example shows that Pyrintegrin treatment with ADM enhanced Adiponectin cytokine secretion in hADSCs.

At two and four weeks post-treatment, Adiponectin content was measured in hADSCs treated with: control medium; adipogenic differentiation medium (ADM); and ADM plus 2 μM of Pyrintegrin (ADM+Drug).

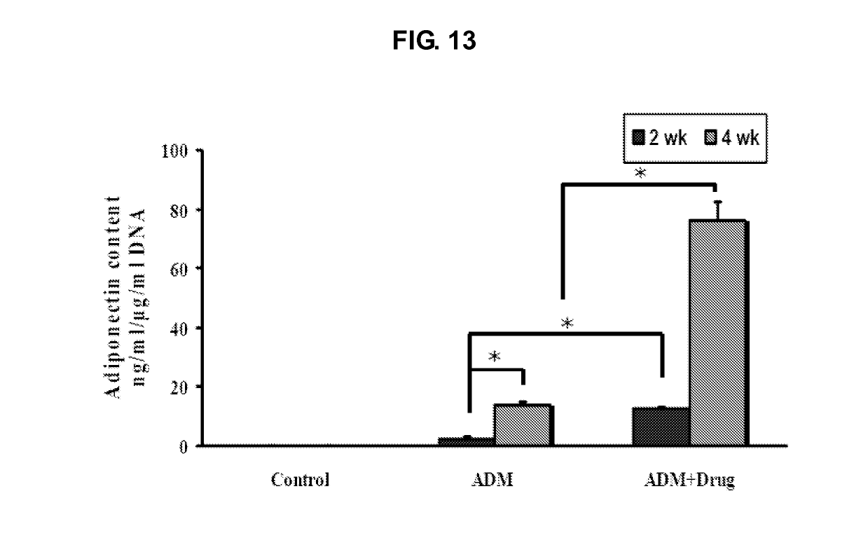

Results showed that Pyrintegrin treatment along with adipogenic medium enhanced the secretion of Adiponectin cytokine in vitro (see e.g., FIG. 13).

### Example 13

This example shows that hADSCs treated with Pyrintegrin and ADM exhibited enhanced secretion of Leptin cytokine in vitro.

At two and four weeks post-treatment, Leptin content was measured in hADSCs treated with: control medium; adipogenic differentiation medium (ADM); and ADM plus 2 μM of Pyrintegrin (ADM+Drug).

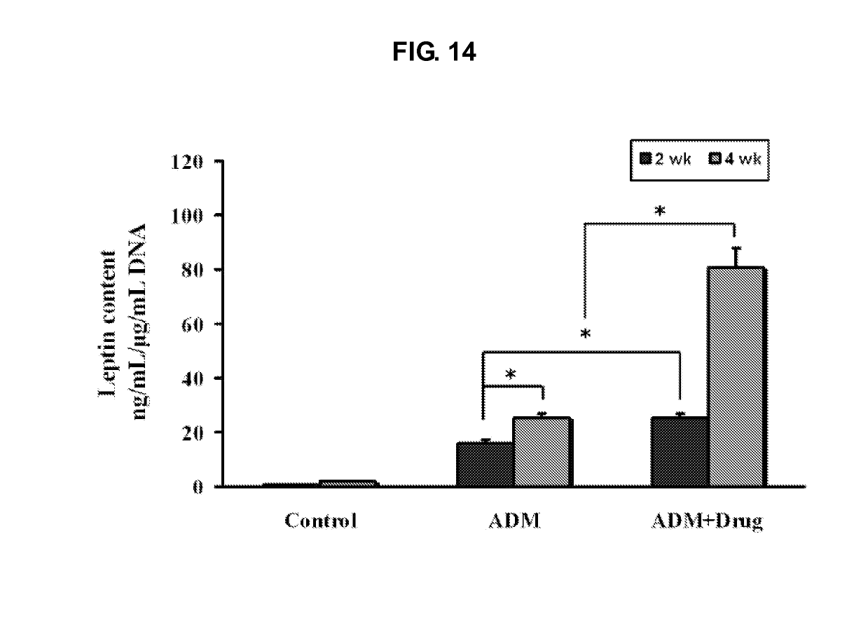

Results showed that Pyrintegrin treatment along with adipogenic medium enhanced the secretion of Leptin cytokine in vitro (see e.g., FIG. 14).

### Example 14

This example shows that hADSCs treated with Pyrintegrin and ADM exhibited enhanced secretion of Glycerol in vitro.

At two and four weeks post-treatment, Glycerol content was measured in hADSCs treated with: control medium; adipogenic differentiation medium (ADM); and ADM plus 2 μM of Pyrintegrin (ADM+Drug).

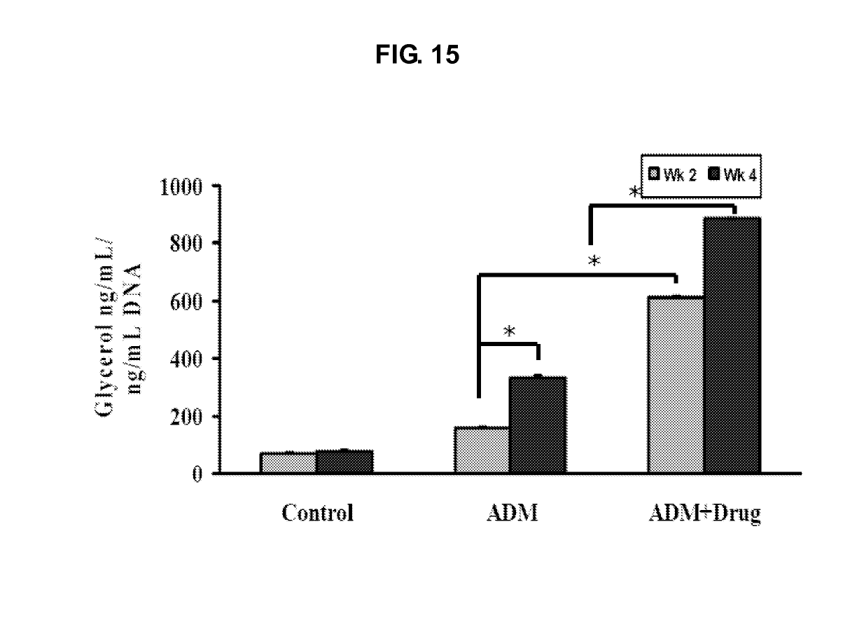

Results showed that Pyrintegrin treatment along with adipogenic medium enhanced the secretion of Glycerol in vitro (see e.g., FIG. 15).

### Example 15

This example shows that Pyrintegrin is a BMP pathway inhibitor as demonstrated by Western blot analysis of hADSCs.

Western blot analysis was performed 1-hour post-treatment in hADSCs treated with: control medium (Control); adipogenic differentiation medium (ADM); ADM plus 2 μM of Pyrintegrin (ADM+Drug); and Pyrintegrin alone (Drug).

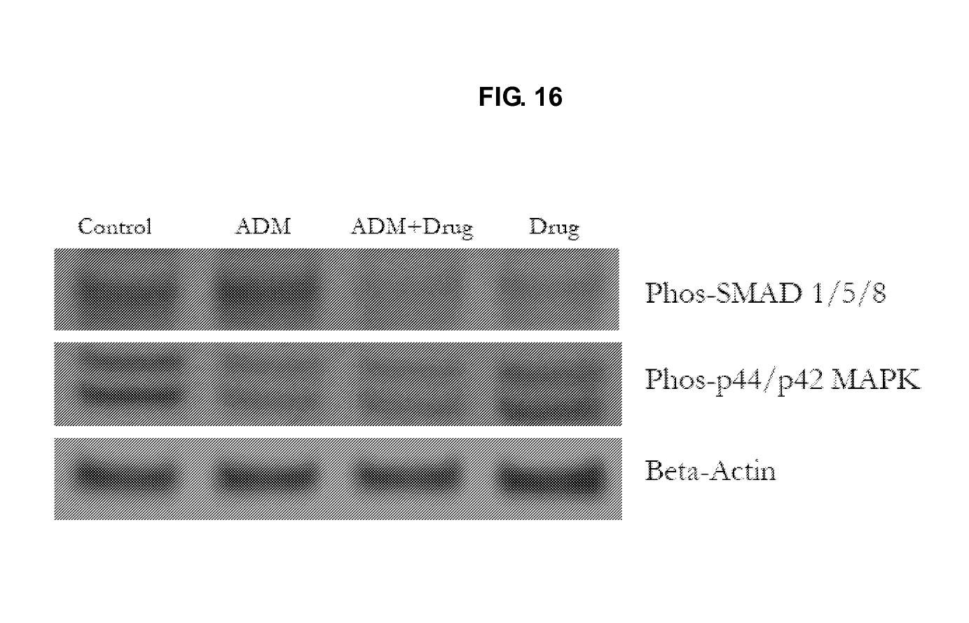

Results showed that Pyrintegrin is a BMP pathway inhibitor as it prevents the phosphorylation of Smad1/5/8 (see e.g., FIG. 16).

### Example 16

This example shows that Pyrintegrin is not a TGFβ/Activin pathway inhibitor.

Western blot analysis was performed 1-hour post-treatment in hADSCs treated with: control medium (Control); adipogenic differentiation medium (ADM); ADM plus 2 μM of Pyrintegrin (ADM+Drug); and Pyrintegrin alone (Drug).

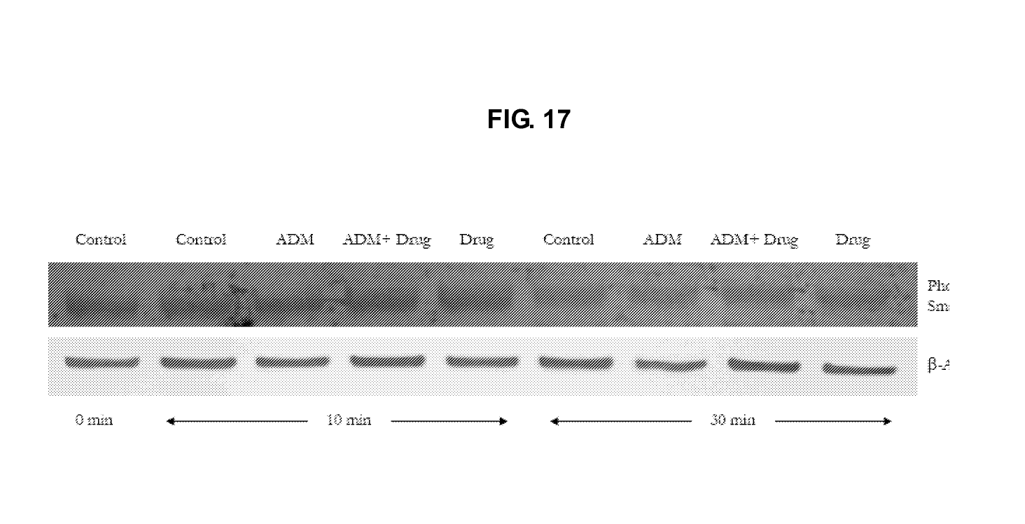

Results showed that Pyrintegrin does not target the TGFβ/Activin pathway (see e.g., FIG. 17).

17. A method of treating a soft tissue defect in a subject, the method comprising:
implanting into a subject in need thereof a scaffold comprising an effective amount of (i) a cell homing composition and (ii) an adipogenic composition;
wherein,
the scaffold does not comprise a transplanted cell, a cell ex vivo, or a cell prior to implantation in the subject;
the effective amount of the cell homing composition induces migration of a progenitor cell into or onto the scaffold, and
the effective amount of the adipogenic composition induces formation of an adipose cell or adipose-like cell from the progenitor cell.

18. The method of claim 17, wherein the cell homing composition comprises:
insulin-like growth factor 1 (IGF1);
basic fibroblast growth factor (bFGF); or
IGF1 and bFGF.

19. The method claim 18, wherein the cell homing composition comprises:
IGF1 at a ratio of about 0.1/250 to about 250/250 (μg IGF1 per mg scaffold); or
bFGF at a ratio of about 0.1/250 to about 250/250 (μg bFGF per mg scaffold).

20. The method of claim 17, wherein the adipogenic composition comprises one or more of indomethacin, insulin, isobutyl-methylxanthine (IBMX), dexamethasone, or Pyrintegrin.

21. The method of claim 20, wherein the adipogenic composition comprises:
indomethacin at a ratio of about 0.1/250 to about 250/250 (mg indomethacin per mg scaffold);
insulin at a ratio of about 0.1/250 to about 250/250 (mg insulin per mg scaffold);
IBMX at a ratio of about 0.1/250 to about 250/250 (mg IBMX per mg scaffold);
dexamethasone at a ratio of about 0.1/250 to about 250/250 (mg dexamethasone per mg scaffold); or
Pyrintegrin at a ratio of about 0.1/250 to about 250/250 (mg Pyrintegrin per mg scaffold).

22. The method of claim 17, wherein the progenitor cell is selected from the group consisting of an adipose tissue derived cell, a pre-adipocyte, a mesenchymal stem cell (MSC), an MSC-derived cell, and an adipocyte.

23. The method of claim 17, wherein the scaffold comprises a biocompatible matrix material.

24. The method of claim 17, wherein the scaffold comprises poly(lactic-co-glycolic acid) (PLGA).

25. The method of claim 17, wherein the scaffold comprises at least one physical channel.

26. The method of claim 17, wherein after migration, the progenitor cells are present in the scaffold at a density of about 0.0001 million cells (M) ml⁻¹ to about 1000 M ml⁻¹.

27. The method of claim 17, wherein after formation, the adipose cells or adipose-like cells are present in the scaffold at a density of about 0.0001 million cells (M) ml⁻¹ to about 1000 M ml⁻¹.

28. The method of claim 17, wherein the scaffold, the cell homing composition, or the adipogenic composition comprises a secretase γ inhibitor, a Notch gamma secretase inhibitor, or a MAPk inhibitor in an amount effect to reduce, substantially reduce, or eliminate adipogenesis inhibition by an EGF receptor comprised of the progenitor cell.

29. The method of claim 28, wherein the scaffold, the cell homing composition, or the adipogenic composition comprises a secretase γ inhibitor.

30. The method of claim 28, wherein the scaffold, the cell homing composition, or the adipogenic composition comprises a secretase γ inhibitor, a Notch gamma secretase inhibitor, or a MAPk inhibitor at a concentration of about 1.0 μM to about 100 μM or at a ratio of about 0.1/250 to about 250/250 (μg inhibitor per mg scaffold).

31. A method of forming adipose tissue comprising:
providing a scaffold comprising an effective amount of (i) a cell homing composition and (ii) an adipogenic composition;
placing the scaffold in fluid communication with a progenitor cell;
inducing migration of the progenitor cell into or onto the scaffold; and
inducing formation of an adipose cell or adipose-like cell from the progenitor cell;
wherein the scaffold does not comprise a transplanted cell, a cell ex vivo, or a cell prior to implantation in the subject.

32. The method of claim 31, wherein at least one of the following is satisfied:
(i) the cell homing composition comprises insulin-like growth factor 1 (IGF1), basic fibroblast growth factor (bFGF), or IGF1 and bFGF;
(ii) the cell homing composition comprises IGF1 at a ratio of about 0.1/250 to about 250/250 (μg IGF1 per mg scaffold) or bFGF at a ratio of about 0.1/250 to about 250/250 (μg bFGF per mg scaffold);
(iii) the adipogenic composition comprises one or more of indomethacin, insulin, isobutyl-methylxanthine (IBMX), dexamethasone, or Pyrintegrin;
(iv) the adipogenic composition comprises indomethacin at a ratio of about 0.1/250 to about 250/250 (mg indomethacin per mg scaffold), insulin at a ratio of about 0.1/250 to about 250/250 (mg insulin per mg scaffold), IBMX at a ratio of about 0.1/250 to about 250/250 (mg IBMX per mg scaffold), dexamethasone at a ratio of about 0.1/250 to about 250/250 (mg dexamethasone per mg scaffold), or Pyrintegrin at a ratio of about 0.1/250 to about 250/250 (mg Pyrintegrin per mg scaffold);
(v) the progenitor cell is selected from the group consisting of an adipose tissue derived cell, a pre-adipocyte, a mesenchymal stem cell (MSC), an MSC-derived cell, and an adipocyte;
(vi) the scaffold comprises a biocompatible matrix material;
(vii) the scaffold comprises poly(lactic-co-glycolic acid) (PLGA);
(viii) the scaffold comprises at least one physical channel;
(ix) after migration, the progenitor cells are present in the scaffold at a density of about 0.0001 million cells (M) ml⁻¹ to about 1000 M ml⁻¹;
(x) after formation, the adipose cells or adipose-like cells are present in the scaffold at a density of about 0.0001 million cells (M) ml⁻¹ to about 1000 M ml⁻¹;
(xi) the scaffold, the cell homing composition, or the adipogenic composition comprises a secretase γ inhibitor, a Notch gamma secretase inhibitor, or a MAPk inhibitor in an amount effect to reduce, substantially reduce, or eliminate adipogenesis inhibition by an EGF receptor comprised of the progenitor cell;
(xii) the scaffold, the cell homing composition, or the adipogenic composition comprises a secretase γ inhibitor; or
(xiii) the scaffold, the cell homing composition, or the adipogenic composition comprises a secretase γ inhibitor, a Notch gamma secretase inhibitor, or a MAPk
